(19) United States
(12) Patent Application Publication
(10) Pub. No.: US 2009/0137505 A1
(43) Pub. Date: May 28, 2009

(54) Method for Predicting and Identifying Target mRnas Controlled By Functional Rnas and Method of Using the Same

(76) Inventors: Roberto Antonio Barrero, West Australia (AU); Takuro Tamura, Shizuoka (JP); Takashi Gojobori, Shizuoka (JP); Kazuho Ikeo, Shizuoka (JP); Tadashi Imanishi, Tokyo (JP)

Correspondence Address: WENDEROTH, LIND & PONACK, L.L.P., 1030 15th Street, N.W.,, Suite 400 East, Washington, DC, 20005-1503, US

(21) Appl. No.: 11/992,261

(22) PCT Filed: Sep. 20, 2006
(86) PCT No.: PCT/JP2006/319097

§ 371 Date: Mar. 19, 2008

(30) Foreign Application Priority Data
Sep. 20, 2005 (JP) 2005-272918

(51) Int. Cl. A61K 31/7088; G06F 19/00; G01N 33/48; C07H 21/00

(52) U.S. Cl. 514/44; 702/20; 536/23.1

(57) ABSTRACT

It is intended to identify or estimate miRNAs and one or more target genes (target mRNAs) targeted thereby.

A method of predicting or identifying miRNAs and one or more target mRNAs targeted thereby, which comprises calculating the most stable secondary structures of double-stranded RNAs, which can be formed by all partial sequences in all of the subject mRNAs with miRNAs, and the secondary structure energies thereof to thereby search for all partial sequences capable of having stable structures through the binding of miRNAs to mRNAs, and then calculating the most stable secondary structure, which can be formed by a subject partial sequence or regions in the vicinity of the subject partial sequence with partial sequences of the concerned mRNA, and the secondary structure energy thereof to thereby determine whether or not the subject partial sequence of the concerned mRNA has a structure capable of interacting with the miRNA.

### TECHNICAL FIELD

The present invention relates to the field of biomolecular regulation. More in detail, the present invention relates to a method for regulating gene expression at the nucleic acid molecules.

### BACKGROUND ART

In October 2001, three groups reported at about the same time the existence of a total of about 100 types of RNA families consisting of small 21-22 bases RNAs among Drosophila, C. elegans, and human (Non-patent documents 1, 2, and 3). These small RNAs were found to regulate the expression of specific mRNAs during the development of organisms by inhibiting the protein synthesis of the targeted mRNAs, and based in their size were named microRNAs (miRNAs). The structural characteristic of miRNA is that precursor RNAs consisting of several tens to several hundreds base pairs form stem-loop structures (shRNA: short hairpin RNA) including double-stranded RNA (dsRNA) regions and that the double-stranded RNA regions have bulge structures including base pair mismatches. It is considered that the precursor miRNAs transferred outside the nucleus are processed into single-stranded mature sequences by Dicer, and then selectively interact mainly with 3′-UTR (untranslated regions) of specific mRNAs to inhibit protein translations (Non-patent document 4).

At present, 2 to 300 types of miRNAs are reported in many organism species including human. The conservation of a number of miRNAs are also reported among a wide variety of species, and databases thereof are being made (Non-patent document 5). Some types of miRNAs are known to have a common nucleotide sequence structure in their nucleotide sequence at the 5′-end (Non-patent document 6), and an attempt has been reported in which nucleotide sequences complementary to these sequences are used as targets in order to search for target mRNAs (Non-patent document 7).

Non-patent document 1: Lagos-Quintana et al, Science 294, 853-868 (2001)
Non-patent document 2: Lau et al., Science 294, 858-862 (2001)
Non-patent document 3: Lee et al., Science 294, 862-864 (2001)

Non-patent document 4: Hutvagner and Zamore, Curr. Opin. Gen. Dev. 12:225-232 (2002)

Non-patent document 5: Griffiths-Jones S., NAR 32, D109-D111 (2004)
Non-patent document 6: Eric C. and Lai, Nature Genetics 30, 363-364 (2002)
Non-patent document 7: Lewis et al., Cell 115, 787-798 (2003)
Non-patent document 8: Reinhart et al., Nature 403, 901-906 (2000)
Non-patent document 9: Zuker et al., Nucl Acid Res 9: 133-148 (1981)
Non-patent document 10: “RNAi Jikken Protocol (RNAi Experimental Protocol) (Jikken Igaku Bessatsu (Special Issue of Experimental Medicine))” p. 95-110 (2003)

### DISCLOSURE OF THE INVENTION

Primary in vivo roles of RNAi and PTGS, which are attracting an attention as short strand RNAs likewise of miRNA, are considered to mainly function as a biological defense system so as to destroy foreign RNAs contaminating through bacterial infection. That is to say, gene expression regulation using this mechanism mainly targets for artificial control points, which may possibly involve unpredicted side effects such as interactions with non target mRNAs.

On the other hand, miRNA is considered to interact in vivo with endogenous target mRNA(s) and thereby regulate the gene expression. The regulation of mRNA expression by miRNA is a naturally-occurring gene regulation mechanism. It is considered that one miRNA may control several mRNAs and also that one type of mRNA may be controlled by various miRNAs. Therefore, if the downstream target(s) for each miRNA can be reliably identified, it then becomes possible to manipulate the in vivo gene expression of the target mRNA(s) by using miRNAs.

Further, applications of experimental approaches based on the information of miRNA molecules and target mRNAs enable the elucidation of roles of target genes in vivo, the development of techniques for treating diseases, and the treatment per se for diseases.

Some research projects are making attempts to search for target mRNAs of miRNAs, for which the approach is to estimate candidate sites on the mRNAs based on the nucleotide sequence pattern and to calculate the optimum secondary structures formed by the miRNA and RNA molecules at the concerned candidate sites and the secondary structure energies thereof so as to examine their adequacy. However, the complementary patterns of nucleotide sequences between known miRNAs and targeted mRNAs are known to be ambiguous, which results in a drawback given that a large amount of similar nucleotide sequence patterns of mRNAs lead to a huge number of predicted target candidates.

The present invention provides a method for predicting or identifying miRNAs and one or more target mRNAs targeted thereby, which comprises calculating the most stable secondary structures of double-stranded RNAs, which can be formed by all partial sequences in all of the subject mRNAs with miRNAs, and the secondary structure energies thereof to thereby search for all partial sequences capable of having stable structures through the binding of miRNAs to mRNAs, and then calculating the most stable secondary structure, which can be formed by a subject partial sequence or regions in the vicinity of the subject partial sequence with partial sequences of the concerned mRNA, and the secondary structure energy thereof to thereby determine whether or not the subject partial sequence of the concerned mRNA has a structure capable of interacting with the miRNA. Further, by confirming that such relations between miRNAs and partial sequences of mRNAs are preserved among different organism species (such as among human-mouse), highly reliable combinations of miRNAs and target mRNAs are obtained.

The present invention provides a method of predicting or identifying protein-encoding genes (target mRNAs) targeted and controlled by the miRNA molecules which are functional RNA molecules capable of regulating gene expressions.

The gene expression of a target mRNA estimated by the present invention can be regulated (protein translation can be regulated) by the miRNA.

The present invention enables to provide expression regulatory agents comprising miRNA as an active ingredient for regulating a target gene expression, and medicaments comprising the expression regulatory agent.

Moreover, the present invention can be used for the development of treatments of diseases associated with proteins encoded by estimated target mRNAs and treatments for diseases associated with proteins encoded by estimated target mRNAs.

This description includes part or all of the contents as disclosed in the description of Japanese Patent Application No. 2005-272918, which is a priority document of the present application.

### BRIEF DESCRIPTION OF THE DRAWINGS

FIG. 1 shows a pattern of the secondary structure energy formed by 3′-UTR of Lin-41 and Let-7.

FIG. 2-1 shows a list of known miRNAs preserved among human and mouse.

FIG. 2-2 shows a list of known miRNAs preserved among human and mouse.

FIG. 2-3 shows a list of known miRNAs preserved among human and mouse.

FIG. 2-4 shows a list of known miRNAs preserved among human and mouse.

FIG. 3 shows a flowchart of a protocol for searching for target mRNAs of miRNAs.

FIG. 4-1 shows a list of miRNAs and estimated target mRNAs thereof.

FIG. 4-2 shows a list of miRNAs and estimated target mRNAs thereof.

FIG. 4-3 shows a list of miRNAs and estimated target mRNAs thereof.

FIG. 4-4 shows a list of miRNAs and estimated target mRNAs thereof.

FIG. 4-5 shows a list of miRNAs and estimated target mRNAs thereof.

FIG. 4-6 shows a list of miRNAs and estimated target mRNAs thereof.

FIG. 4-7 shows a list of miRNAs and estimated target mRNAs thereof.

FIG. 4-8 shows a list of miRNAs and estimated target mRNAs thereof.

FIG. 4-9 shows a list of miRNAs and estimated target mRNAs thereof.

FIG. 4-10 shows a list of miRNAs and estimated target mRNAs thereof.

FIG. 4-11 shows a list of miRNAs and estimated target mRNAs thereof.

FIG. 4-12 shows a list of miRNAs and estimated target mRNAs thereof.

FIG. 4-13 shows a list of miRNAs and estimated target mRNAs thereof.

FIG. 4-14 shows a list of miRNAs and estimated target mRNAs thereof.

FIG. 4-15 shows a list of miRNAs and estimated target mRNAs thereof.

FIG. 4-16 shows a list of miRNAs and estimated target mRNAs thereof.

FIG. 5 shows combinations between miRNAs, target mRNAs, and subject proteins verified by experiments.

FIG. 6 shows secondary structure energies in the combinations between miRNAs and target mRNAs verified by experiments.

FIG. 7 shows partial sequences of mRNAs and modified sequences thereof used for experiments.

FIG. 8 shows experimental results 1: Dual Luciferase assay.

FIG. 9 shows experimental results 2: RT-PCR and Western blotting.

FIG. 10 shows experimental results 3 (quantification of the results of FIG. 9).

### BEST MODE FOR CARRYING OUT THE INVENTION

Functional RNA molecules in the present invention include 16 to 25 bases-long RNA molecules having a gene expression regulatory activity, and specifically miRNAs.

MiRNA molecules serving as subjects of the present invention may be naturally-occurring ones in any animal such as human, mouse, rat, chicken, zebrafish, C. elegans, and Drosophila. Moreover, artificially designed miRNA molecules targeting a specific organism may also serve as subjects.

[Identification of Subject Genes Controlled by miRNAs]

The method of predicting or identifying genes controlled by miRNAs (target mRNAs) of the present invention comprises the following first step, second step, and third step.

The first step is a step of calculating, among sets of miRNAs and target mRNA candidates, the structure energies in partial sequences of mRNAs with miRNA sequences to search for stably-bindable partial sequences, so as to select sets of miRNAs and mRNAs having such partial sequences.

The second step is a step of calculating, among sets of miRNAs and mRNAs selected in the first step, the most stable secondary structure energy, which can be formed by the partial sequence of the mRNA that is stably-bindable with the miRNA or sequences in the vicinity thereof within the mRNA, to thereby search for partial sequences incapable of forming a stable structure within the mRNAs, so as to select sets of miRNAs and mRNAs having such partial sequences.

Since it is considered that one type of miRNA may control a plurality of types of mRNAs and that one type of mRNA may also be controlled by a plurality of types of miRNAs, the first and second steps can be respectively repeated to search for partial sequences by changing target mRNA candidates, until all sets of mRNAs and miRNAs meeting the criteria are found out.

The third step is a step of performing the first step and the second step using a different organism species as a subject so as to select, with respect to miRNAs having a nucleotide sequence structure preserved among organism species, mRNAs selected as target mRNAs concerning each organism species similarly having a nucleotide sequence structure preserved among organism species, that is to say, sets in which the binding environments formed by miRNAs and partial structures of mRNA sequences have been preserved among organism species.

RNA secondary structure calculation algorithm is desirably used for calculating the structure energy in the first step. This is because that pairs of nucleotide sequences formed by bindings between miRNAs and mRNAs are not completely matched but are known to be ambiguously bound including gap(s), and that mere calculation of energies of complementary nucleotide sequences would not provide desired structure energies. For the same reason, a partial sequence of mRNA serving as a subject of calculation desirably have a different length from the length of miRNA and is an approximately 3 to 8 bases-longer region. For example, regarding this partial sequence, partial sequences may be serially selected from an end, preferably the 3′-end, of mRNA, in a length of 3 to 8 bases-longer than the length of miRNA as subjects of calculation. Moreover, as required, target mRNA candidates searched by other prediction methods or partial sequences in the target mRNA candidates may be selected as subjects of calculation in the first step. For the RNA secondary structure calculation algorithm, general RNA secondary structure calculation algorithms may be used. For example, the calculation can be readily achieved by those described in Non-patent document 9, various programs developed on the basis of Non-patent document 9, and program libraries (for example, Vienna RNA package: http://www.tbi.univie.ac.at/˜ivo/RNA/). Further, it is effective to speed up the calculation by limiting the subjects of calculation within secondary structures which can be formed by double-strands of short RNAs. That is to say, hairpin loops, tetraloops, triloops, multibranch loops, and the like can be excluded from the calculation algorithm since stable structures of double-strands between miRNAs and mRNAs serving as search subjects are not satisfied, even if the formation thereof is possible. For example, the calculation of binding energy may be speeded-up by considering only Watson-Click base pairs, G-U wobble base pairs, bulge loops, and internal loops.

It is obvious to those skilled in the art what sort of conditions should be applied to stably-bindable partial sequences to be found out by searching through the calculation of secondary structure energies in partial sequences of mRNAs with miRNA sequences. For example, when parameters of the above Vienna RNA package are used, the condition would be −18.0 Kcal to −22.0 Kcal or less, which also includes lower values than secondary structure energies of Watson-Click structures (double-stranded complementary structures) formed between 7-bases poly(C) and 7-bases poly(G).

As to miRNA sequences to be used in the present invention, any miRNA sequences which have been already collected in various databases may be used. Moreover, as to gene mRNA candidates serving as targets of control by a certain miRNA, since a large number of gene sequences (cDNAs) concerning cDNAs corresponding to mRNAs are already archived in databases, cDNA sequences archived in any database such as DDBJ, EBI, and NCBI may be used. In particular, cDNA sequences archived in RefSeq, which is a cDNA sequence database established by NCBI (The U.S. National Center for Biotechnology Information, hereunder can be abbreviated as NCBI), may be preferably used.

In the search for target mRNA candidates of miRNAs in the first step, the calculation time can be reduced by focusing the scope of searching for interactive sites on target mRNAs within the 3′-UTR regions thereof, because it is considered that there is less space for encoding 5′-UTRs including cis elements such as translation initiation signals and functional sequences for translational regulation. Specifically, cDNAs having 3′-UTR are extracted from collected cDNA sequences. Then, concerning the extracted cDNAs, respective regions from the initiation site of 3′-UTR to the cDNA tail can be set as sequences of search subjects.

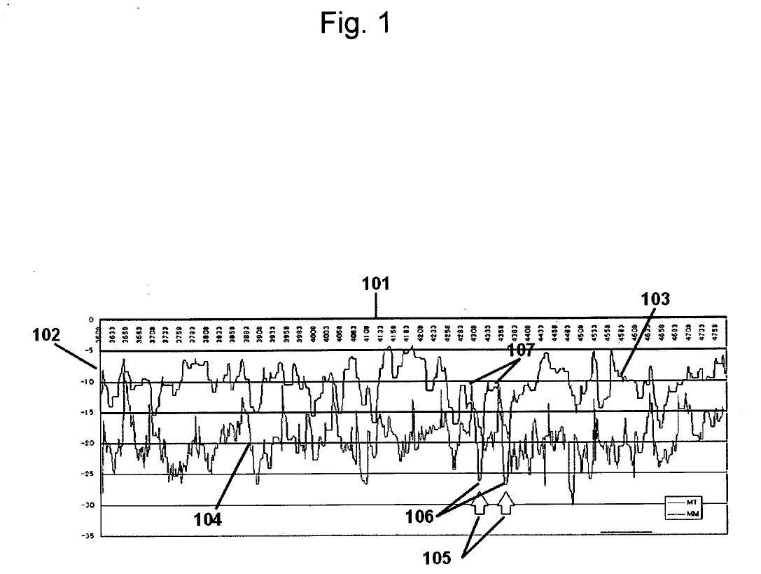

The reason for searching for partial sequences incapable of forming a stable structure within mRNA in the second step is based on the assumption that miRNA readily acts on partial sequences incapable of forming a stable structure within mRNA. This is an invention based on the discovery in sets of Let-7 and Lin-41 that are pairs of already known miRNA and mRNA. The fact that Let-7 acts on Lin-41 to inhibit the translation thereof is the earliest reported instance of miRNA (Non-patent document 8), and Let-7 is known to interact with 3′-UTR of Lin-41. FIG. 1 is a line graph showing calculation results using parameters of the Vienna RNA package as parameters of RNA secondary structure energy, wherein the horizontal axis (101) is set to the position on 3′-UTR sequence of Lin-41 and the vertical axis (102) is set to the energy value of secondary structure, which shows energy values of secondary structures which can be formed by partial sequences on 3′-UTR sequence of Lin-41 with Let-7 (103) and energy values of secondary structures which can be formed by partial sequences within Lin-41 (104). Two points indicated by the arrows (105) are Let-7 binding sites on the nucleotide sequence of 3′-UTR. Since the secondary structure energies that can be formed between Let-7 and Lin-41 are very low (106) and conversely the secondary structure energies that can be formed within Lin-41 are high (107), it was revealed that more stable binding is readily possible between Let-7 and Lin-41. Let-7 and partial sequences of Lin-41 suggest a possibility in which the Let-7 binding site may be possibly present in the vicinity of a part which hardly forms a secondary structure within Lin-41.

In the second step, when partial sequences incapable of forming a stable structure within mRNAs are to be searched for, such structures can be searched by searching for partial sequences incapable of forming a stable structure within mRNAs, among partial sequences in the vicinity of 0 to 20 bases-shifted positions from partial sequences that have been searched in the first step. Eventually, in the second step, partial sequences such that the most stable secondary energies which can be formed by these partial sequences or sequences in the vicinity thereof within the mRNAs, are relatively high, and that are stably-bindable with the miRNAs having low secondary structure energies between miRNAs and mRNAs, are selected. In this case, also, by setting the search criterion of partial sequences in the second step as the relative value with respect to secondary structure energy with miRNA, the judgment can be facilitated. For example, the most stable secondary structure energies, formed by partial sequences having low structure energies with miRNAs, or sequences in the vicinity thereof, with other partial sequences within the mRNAs, are calculated, and then cases where the resultant values are higher by 5 kcal or more than secondary structure energies which can be formed by the partial sequences that are stably-bindable with the miRNAs through the binding to the miRNAs, can be selected.

In the third step, using sequences archived in publicly known databases, calculations may be performed concerning at least one type of species other than organism species examined in the first and second steps. Moreover, the third step may also be performed by examining whether or not sets of miRNAs and mRNAs in other species corresponding to sets of miRNAs and mRNAs in a certain species selected in the second step satisfy the criteria of the second step.

Here, miRNAs in other species corresponding to miRNAs in a certain species may be obtained by using, for example, “The miRNA Registry (http://www.sanger.ac.uk/Software/Rfam/mirna/index.shtml)” Nucleic Acids Research, 2004, Vol. 32, Database Issue, D109-D111 which is an miRNA resource (including information on miRNAs preserved among organisms).

Moreover, corresponding mRNA in other species may be obtained by using (1) “Homologene (http://www.ncbi.nlm.nih.gov/entrez/query.fcgi?db=homologene)” (Nucleic Acids Research, 2001, Vol. 29, No. 1 137-140, that is a database of genes preserved among species ranging over various organisms produced by The U.S. National Center for Biotechnology Information (NCBI)) or (2) “The Mouse Genome Database (http://nar.oupjournals.org/cgi/content/full/33/suppl_{—}1/D471)” (Nucleic Acids Research, 2005, Vol. 33, Database issue D471-D475, that is a database of mouse having data of genes preserved among human-mouse), which are resources of mRNAs preserved among organisms.

Moreover, the present method may be used in combination with other identification and prediction methods. Further, after the identification or the prediction by the present method, the confirmation can be also performed by actually transfecting miRNA into cells through experiments, followed by examination on whether or not the expressions of mRNAs estimated by the present method are influenced.

Further, in order to examine whether or not the expressions of target mRNA candidates are inhibited by miRNA, influences on the expressions of the target mRNA candidates can be examined by detecting, in both cases where miRNA has been transfected/not transfected into cells, the target mRNA candidates or corresponding cDNAs in these cells, using a DNA/RNA chip on the surface of which a plurality of the target mRNA candidates or complementary strands thereof are arranged.

[How to use miRNA]

### [Target Gene Expression Suppressing Agent]

If target genes whose expressions are controlled by miRNA are identified, the expressions of these target genes can be controlled using the miRNA, and the miRNA can be used as an expression regulatory agent of these target genes.

For example, besides the transfection of an miRNA into cells as it is, an expression vector producing the miRNA can be prepared. Target gene expression inhibitors may comprise an miRNA or an miRNA expression vector, and if necessary, other additives effective for the transfection into a subject organism, such as calcium phosphate, lipofeline, polylysine, and other additives.

For the preparation of the miRNA expression vector, gene recombination techniques usually employed for the subject organism species may be used. Methods using siRNA expression vector may be used for general animals as subjects. As to the system for expressing siRNA, an RNA polymerase III can be used, which includes a tandem type and a stem-loop type. For example, piGENE™ hU6 and piGENE™ tRNA (iGENE Therapeutics) may be used. Preferably, (i) a tandem type can be made by amplifying siRNA corresponding to selected miRNA with primers including sense- and antisense-sequences, cutting amplified fragments with restriction enzymes, and inserting into the downstream of the U6 promoter, or (ii) oligonucleotides including sense-, loop-, and antisense-sequences can be annealed and inserted into the downstream of the U6 promoter (Non-patent document 10). The expression vector can be transfected into cells by using a kit for cellular transfection such as Effectin™.

The prepared miRNA or siRNA expression vector is transfected into cells or organisms by publicly known methods such as the electroporation, the ca+ polyphosphate method, and the particle gun method.

Next, it is confirmed whether or not the expressions of thus specified control subject genes are actually inhibited by the transfection of miRNA. If functions of the control subject genes are not confirmed, changes in phenotypes resulting from the inhibition are confirmed.

[Design of Artificial miRNA for Target Sequence]

The present invention enables to select target sites (interactive sites) in target mRNAs when an artificial miRNA is produced. For example, miRNA can be designed using as a subject, for example, a region incapable of forming a stable secondary structure on 3′-UTR of mRNA whose gene expression is desired to be controlled, within the mRNA, regions in the vicinity thereof, or regions on the 3′-end thereof. In this case, artificial miRNA can be designed by inserting an optional number of mutations into RNAs which are complementary to the subject region on the mRNA to form a group of candidate miRNAs, followed by the selection, among them, miRNAs that are selectively and stably bindable to the subject region.

### EXAMPLE 1

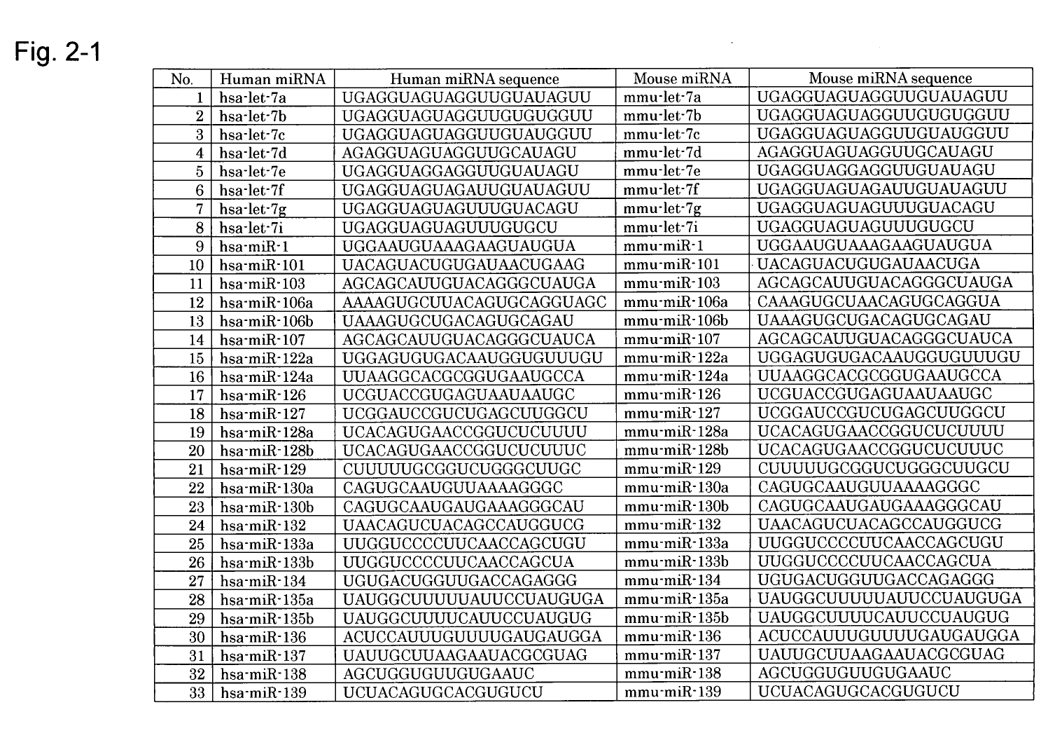

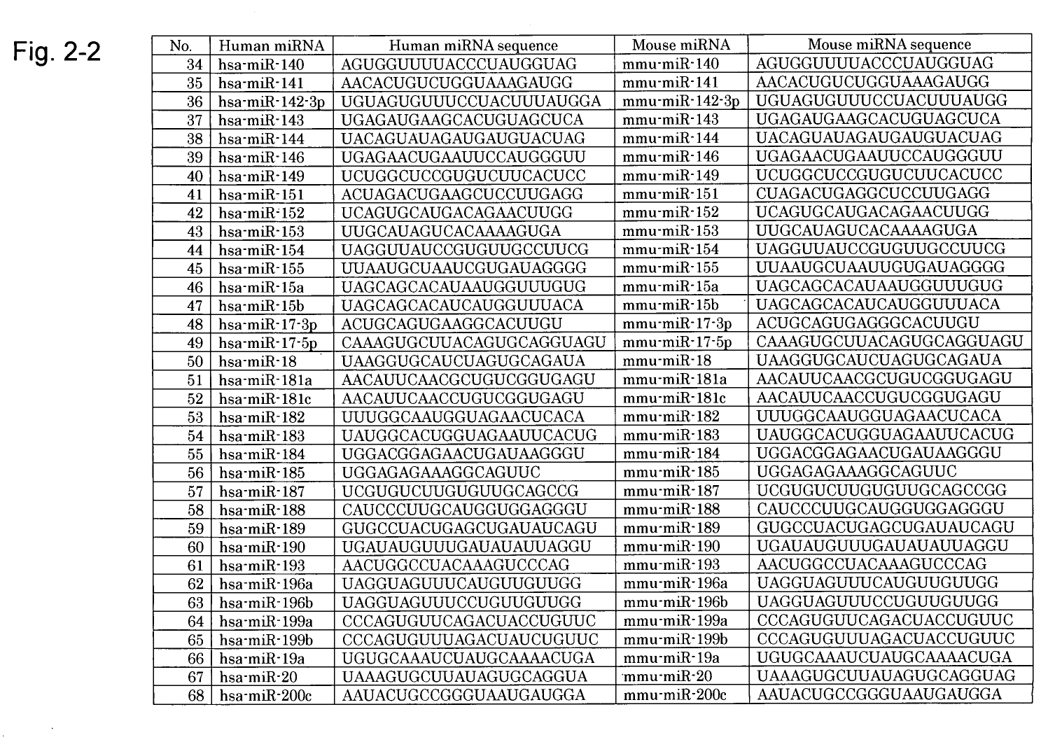

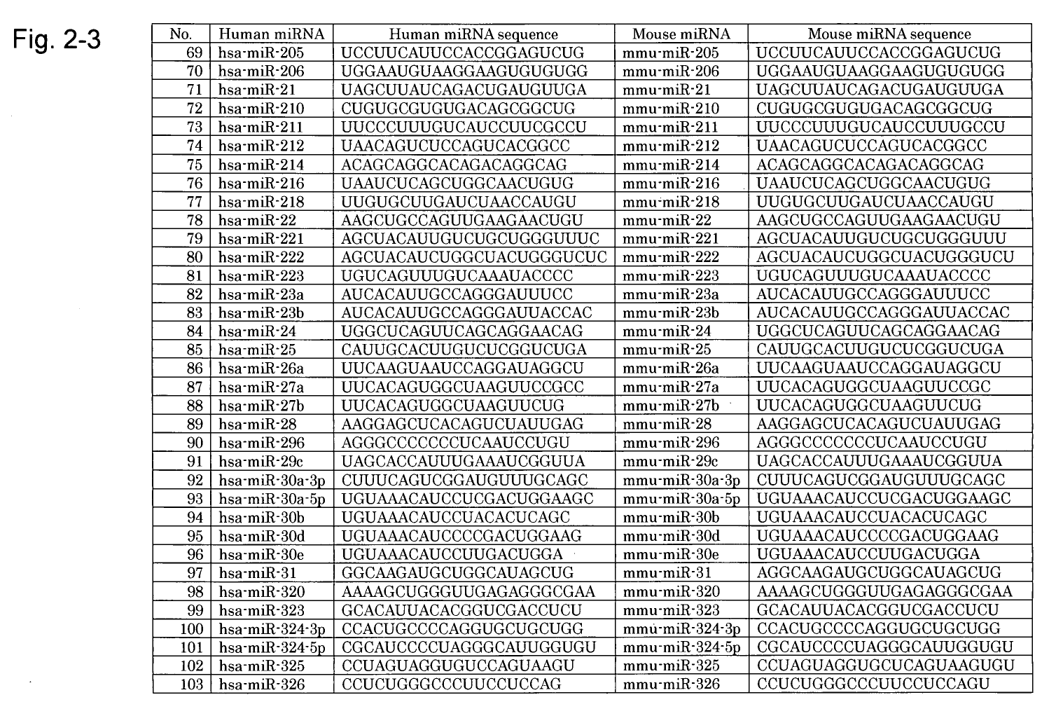

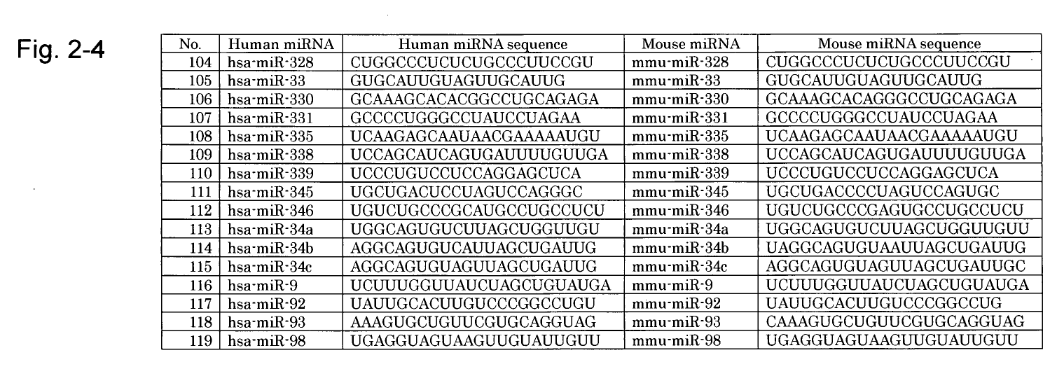

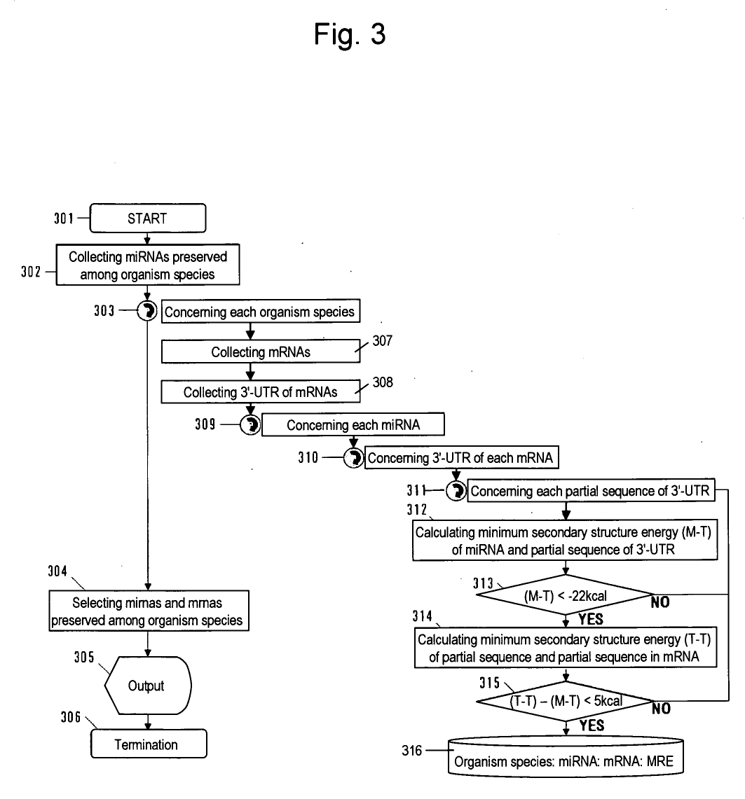

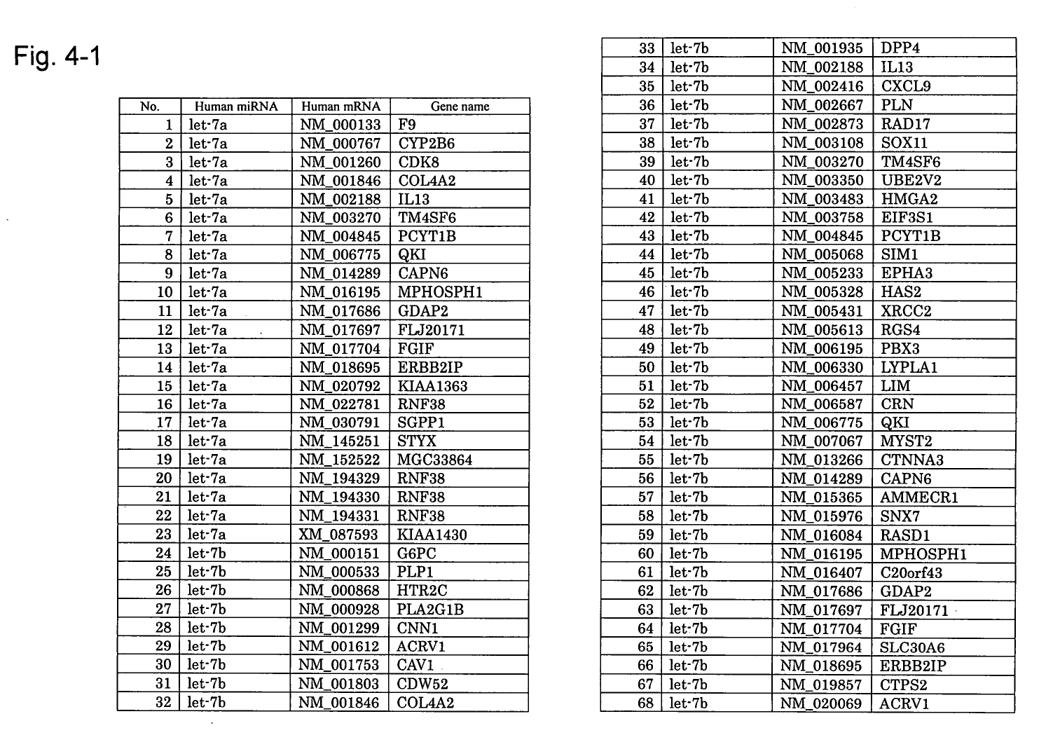

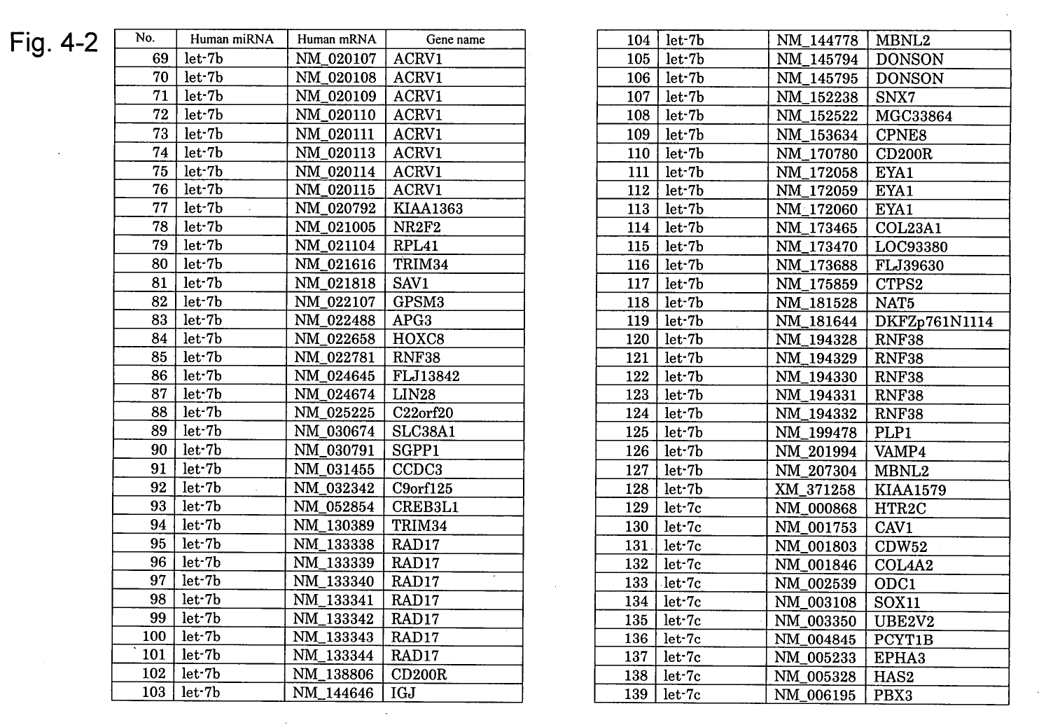

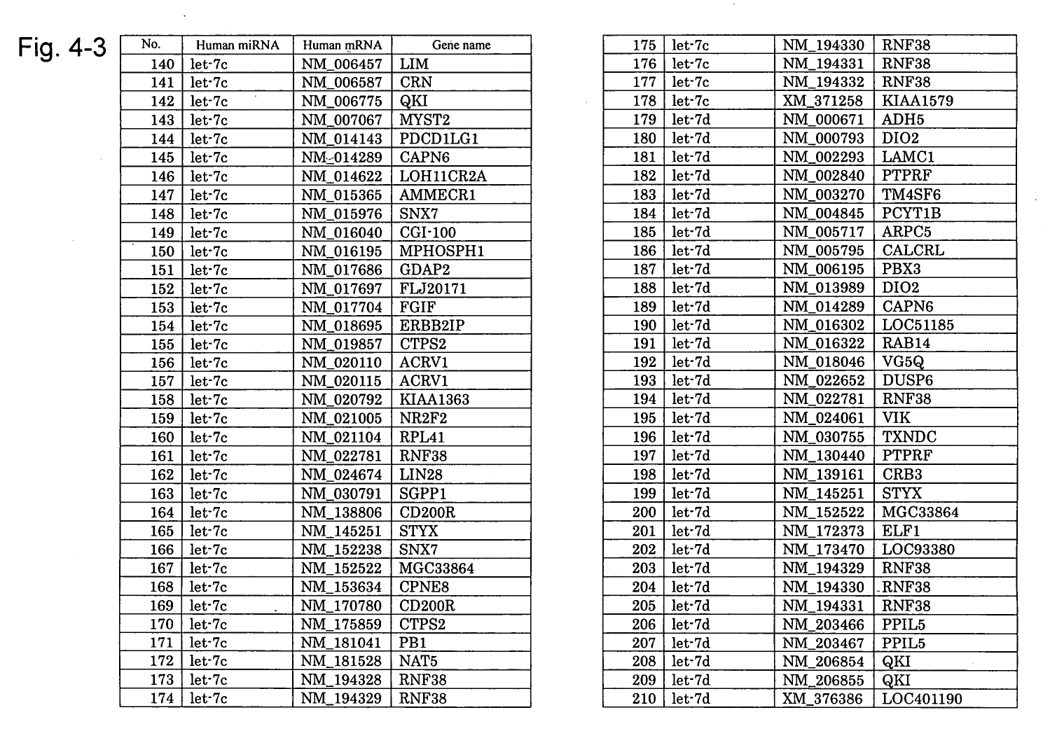

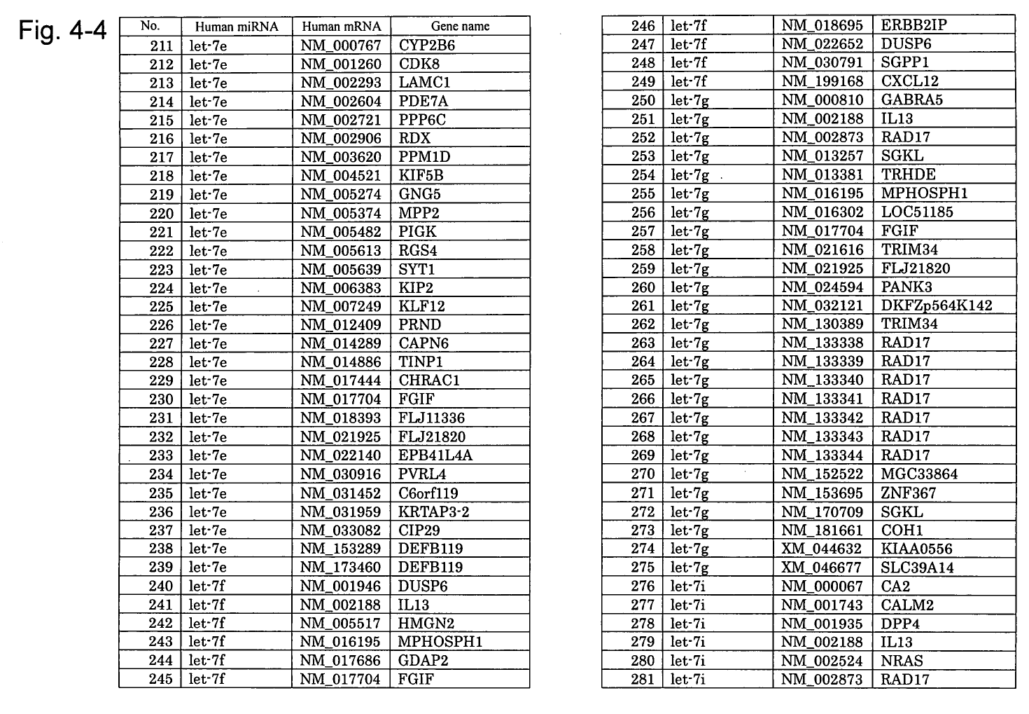

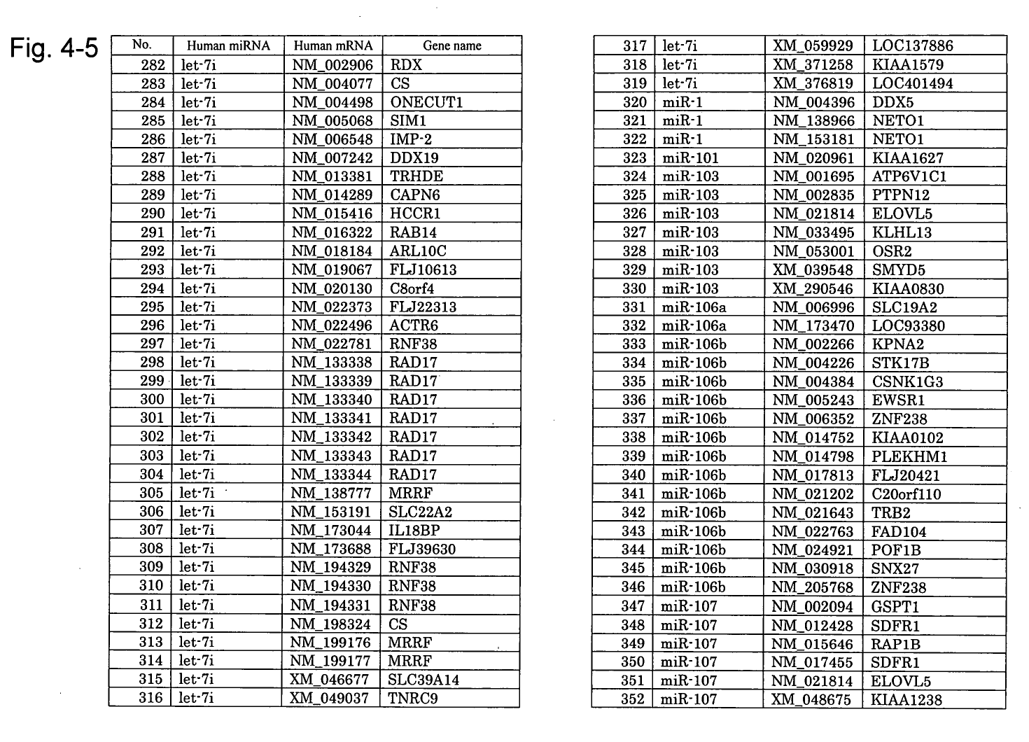

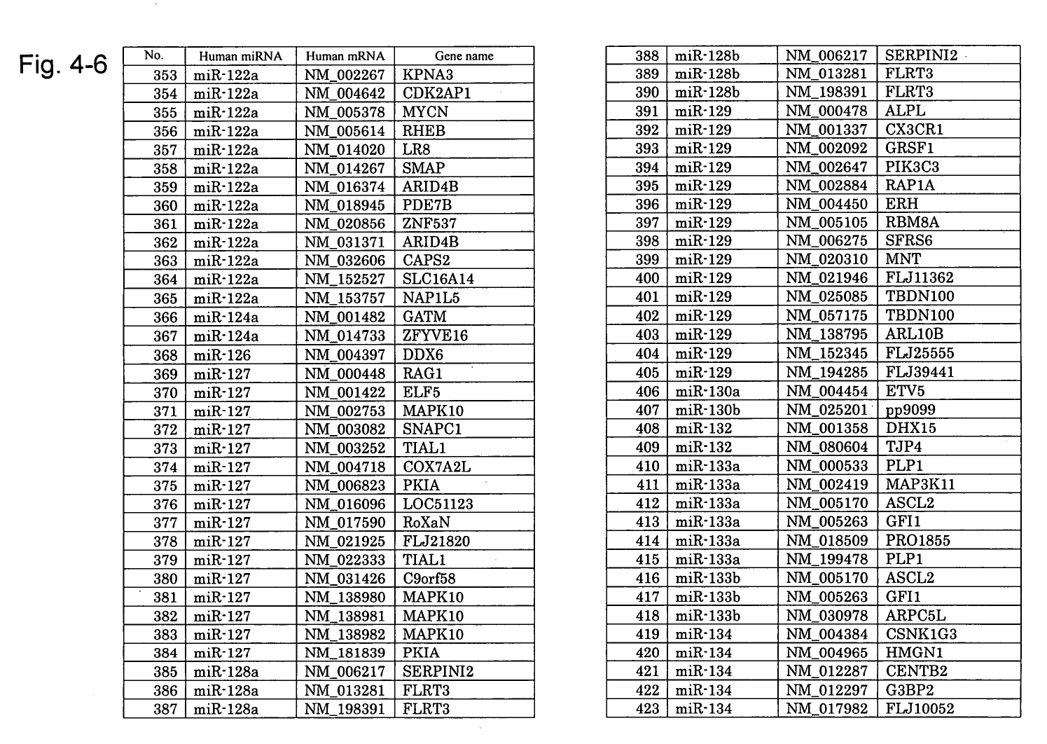

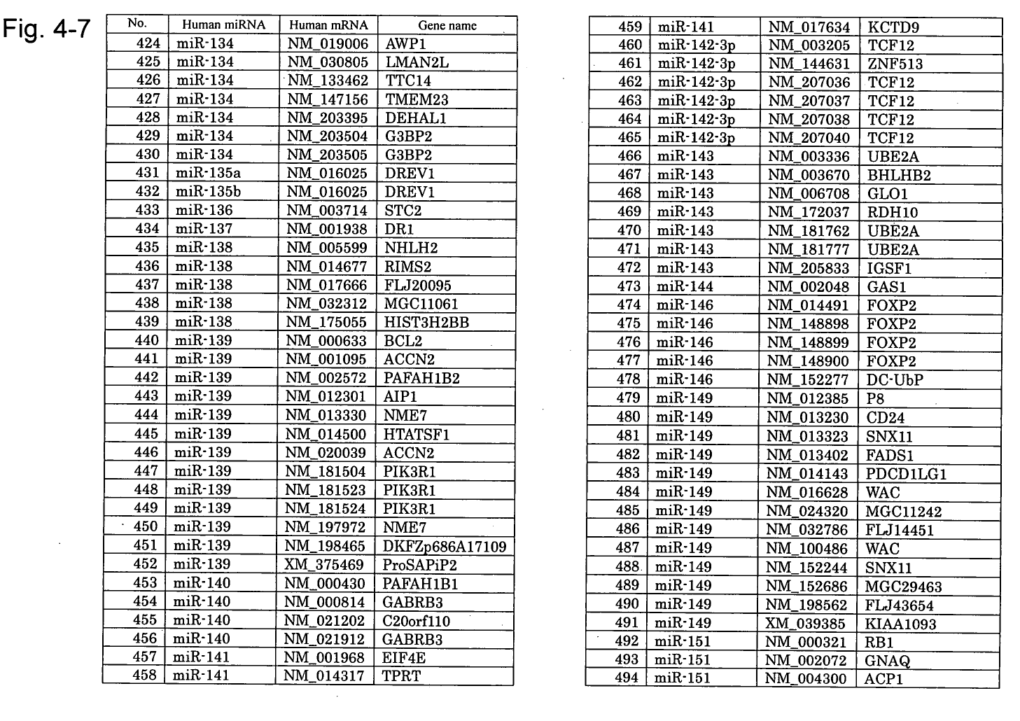

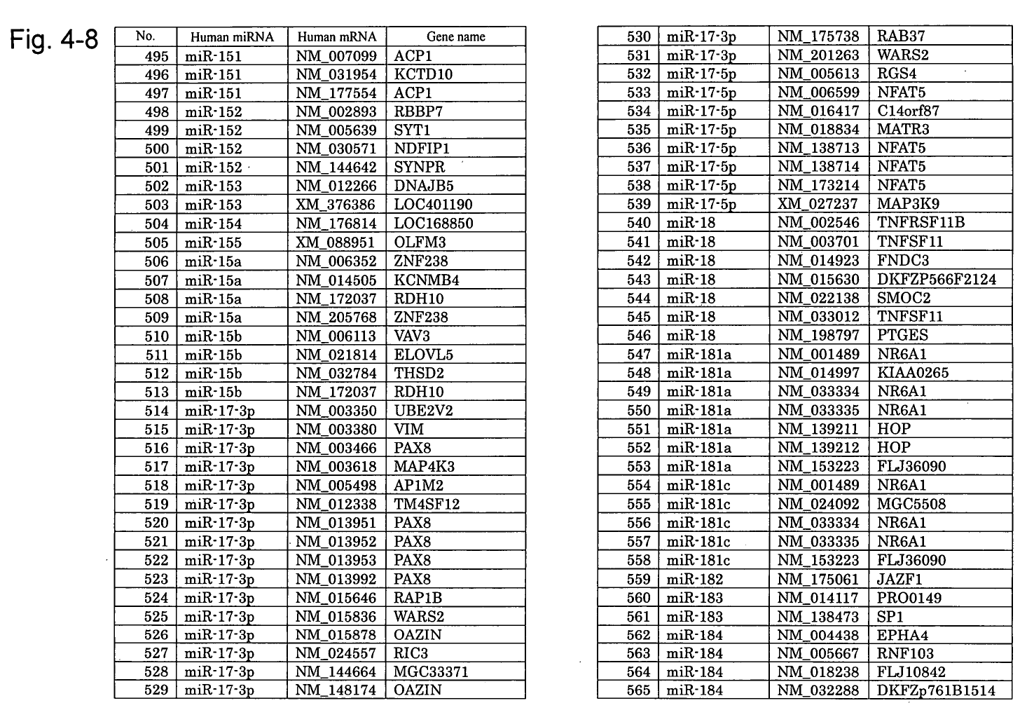

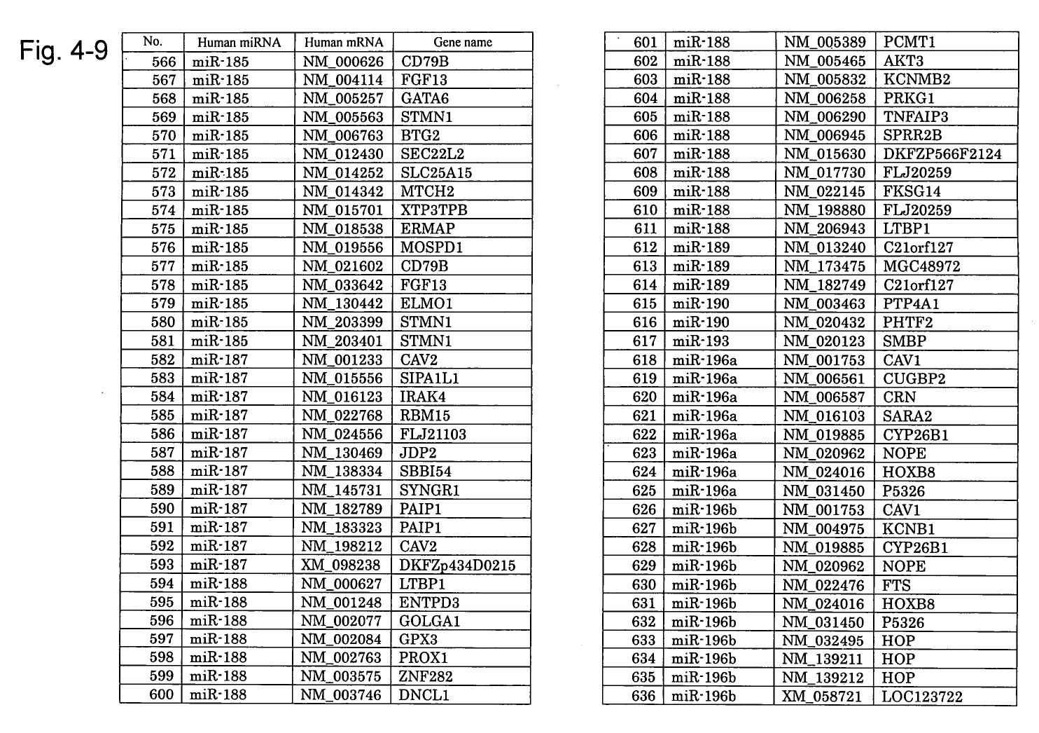

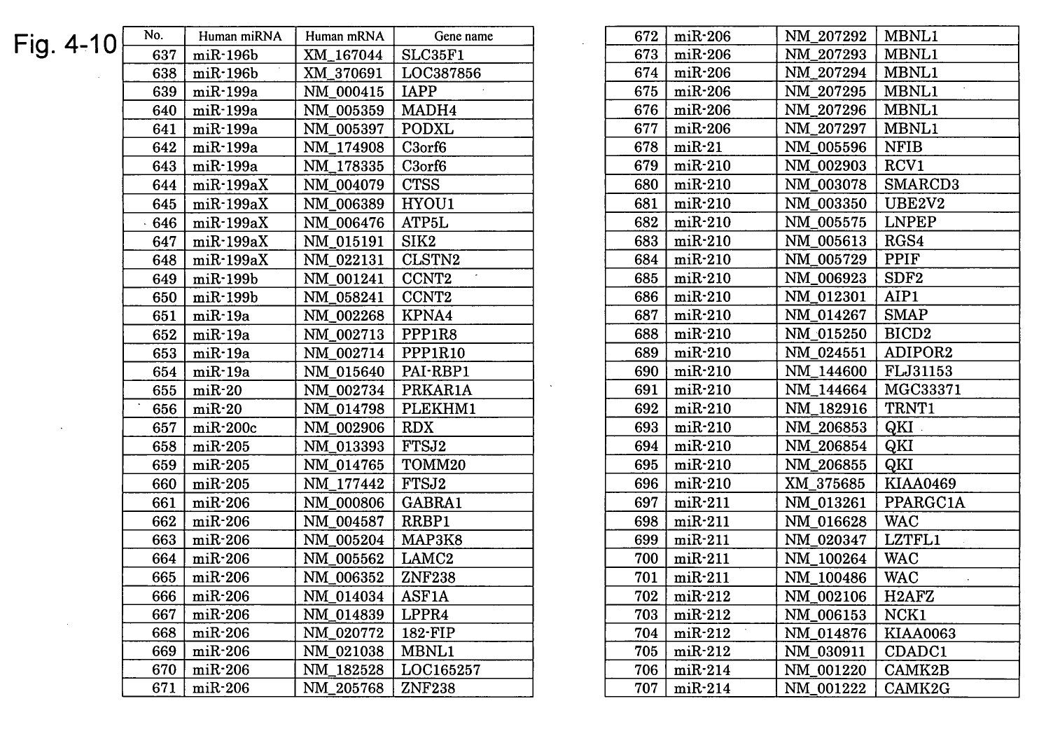

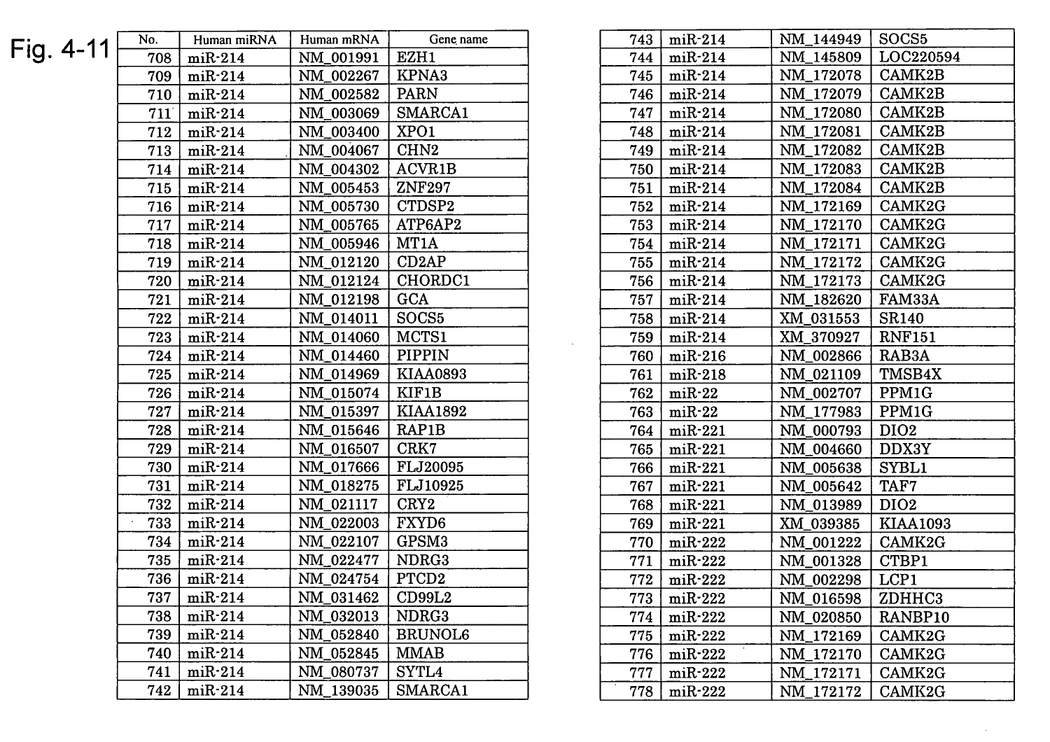

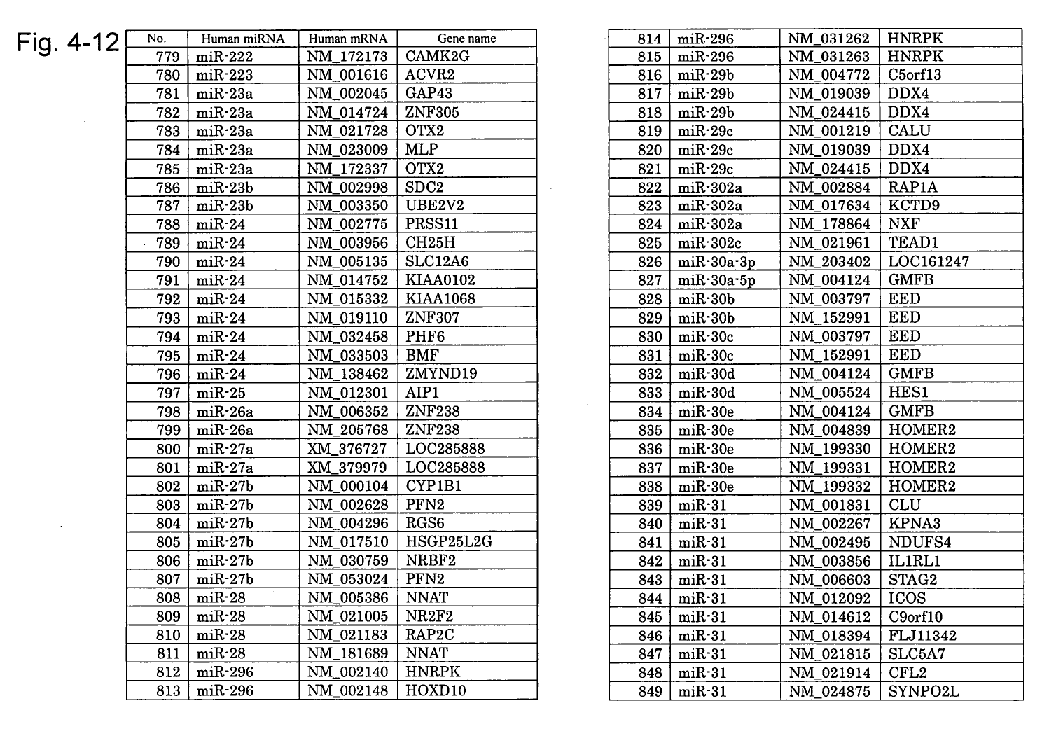

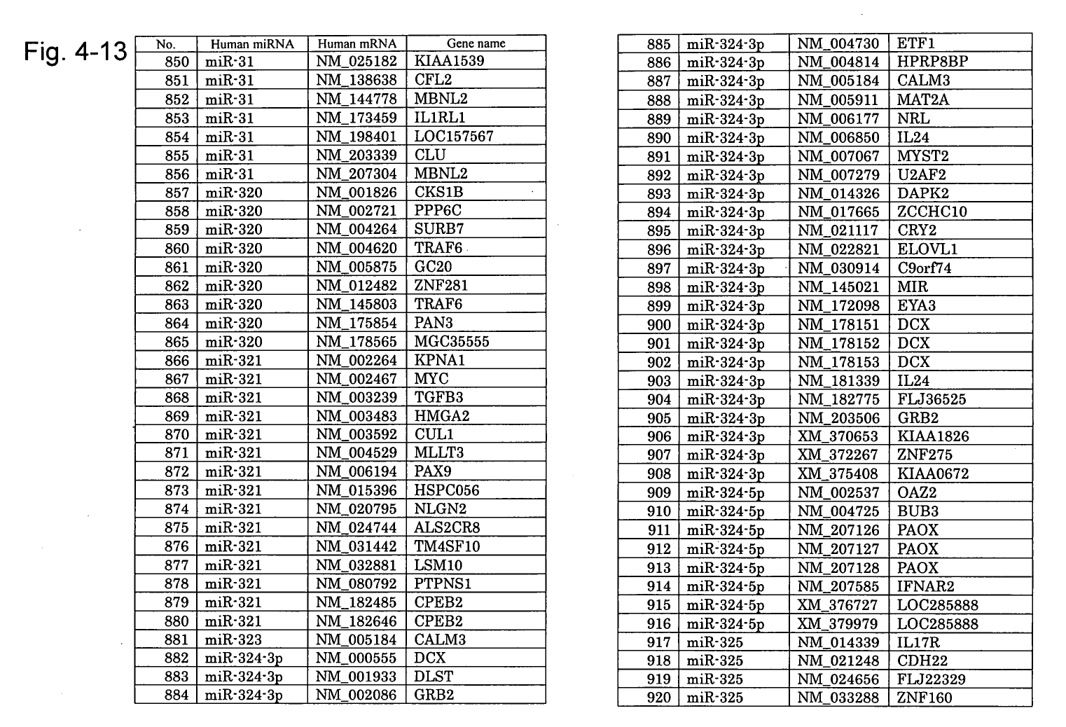

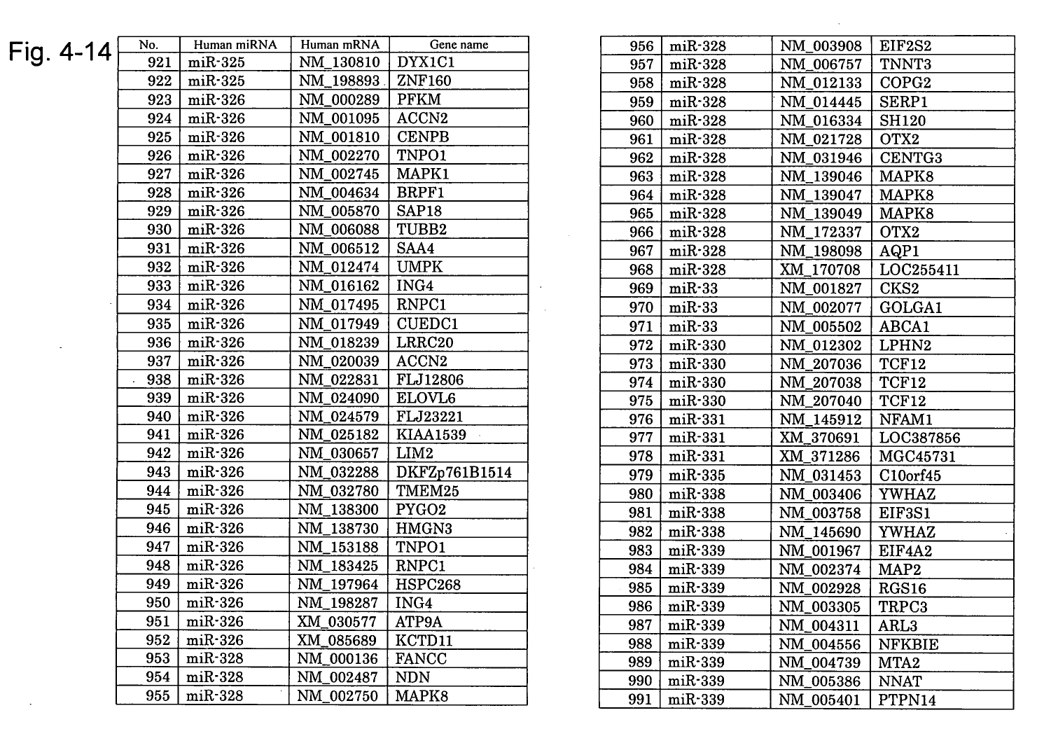

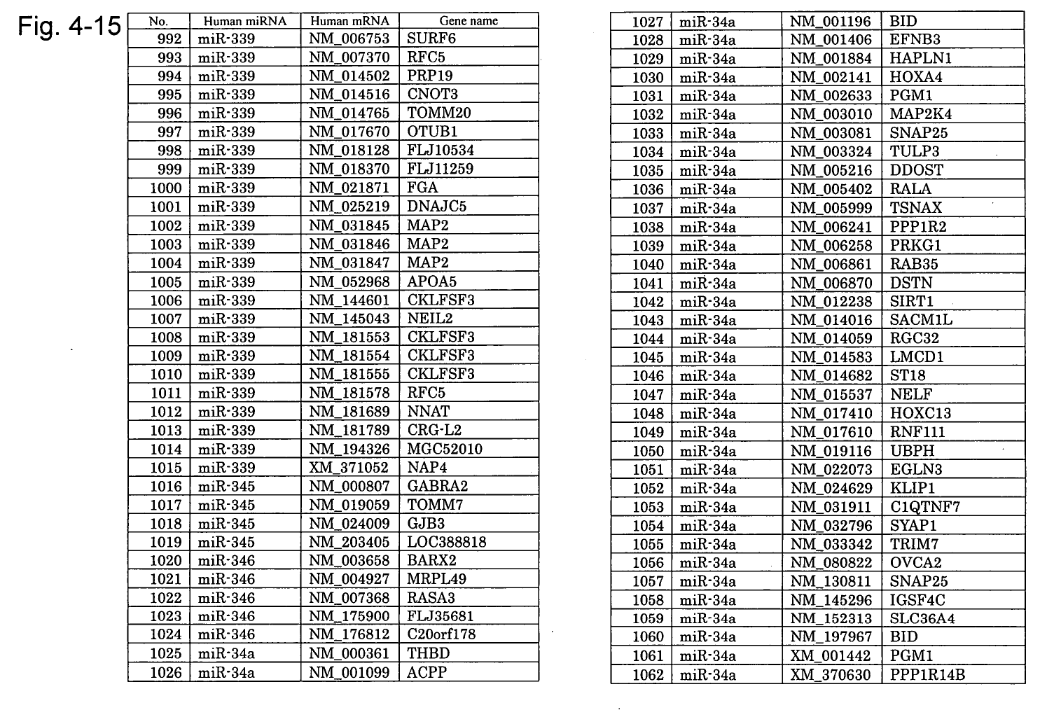

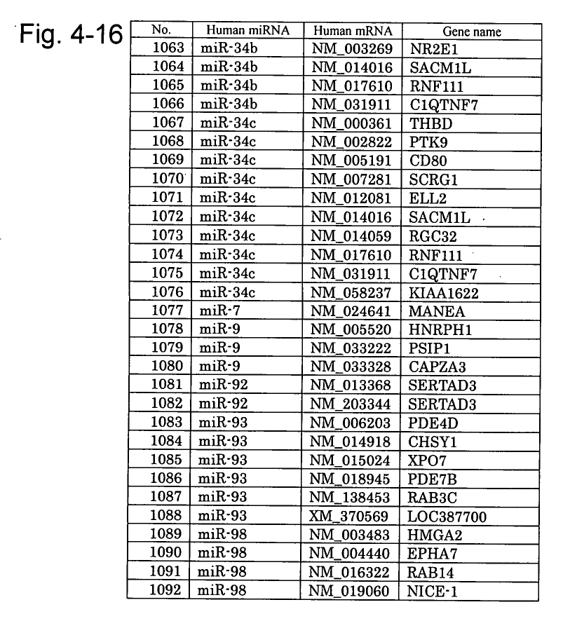

An Example of the search for target mRNAs of miRNAs in the present patent is described in line with the flowchart (FIG. 3). First, 119 types of already-known sets of miRNAs preserved among human and mouse (FIG. 2-1 to FIG. 2-4) were collected from international DNA databanks and research papers (302).

Subsequently, for searching for target mRNA candidates of miRNAs, corresponding target cDNA candidates are searched concerning each organism species (303). The target cDNAs were collected from RefSeq database (Release6) established by Reference Sequence Project of The U.S. National Center for Biotechnology Information, by which 28,176 types of human cDNA sequences and 26,561 types of mouse cDNA sequences were collected (307). Among them, 25,284 types of human cDNA sequences and 19,287 types of mouse cDNA sequences having 3′-UTR were used as target cDNA candidates (308).

For each organism species, concerning each miRNA (309), 3′-UTR of each mRNA (310), and each partial sequence (311), the secondary structure energy between miRNA and partial sequence was calculated (312), and partial sequences capable of forming secondary structures of −22 kcal or less were used as miRNA stably-bindable partial sequences (313). Here, the length of the partial sequences was made 3-bases longer than the length of the nucleotide sequence of subject miRNA.

Subsequently, the most stable secondary structure energies, formed by the miRNA stably-bindable partial sequences with other partial sequences within the miRNAs, were calculated (314). Then, in cases where the resultant values were higher by 5 kcal or more than the secondary structure energies which can be formed by the miRNA stably-bindable partial sequences through the binding to the miRNAs (315), the partial sequences were used as the bindable candidate partial sequences (MRE: miRNA Responsive Element) (316). The above procedures are executed respectively for human and mouse, and combinations of miRNAs having nucleotide sequence structures preserved among human and mouse and mRNAs having nucleotide sequence structures preserved among human and mouse, which respectively have MRE, were collected (304). As a result, combinations of 119 types of human miRNAs and 1,092 types of corresponding human target genes (target mRNAs) were obtained (305). Here, an miRNA family was used for the subject of miRNAs having nucleotide sequence structures preserved among human and mouse, and ortholog information supplied from MGI (Mouse Genome Informatics: http://www.informatics.jax.org/) was used for the subject of mRNAs having nucleotide sequence structures preserved among human and mouse.

The identifiers (such as NM_{—}001949, NM_{—}020390, and NM_{—}004496) representing mRNAs in FIG. 4-1 to FIG. 4-16 are the mRNA registration numbers (accession numbers) given by RefSeq of NCBI, for which http://www.ncbi.nlm.nih.gov/entrez/query.fcgi?db=Nucleotide can be referred to.

### EXAMPLE 2

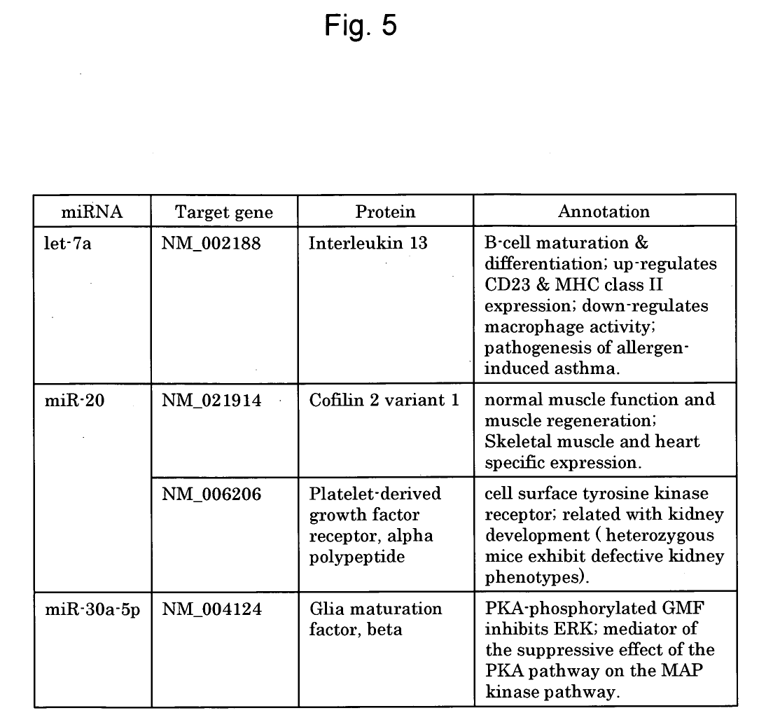

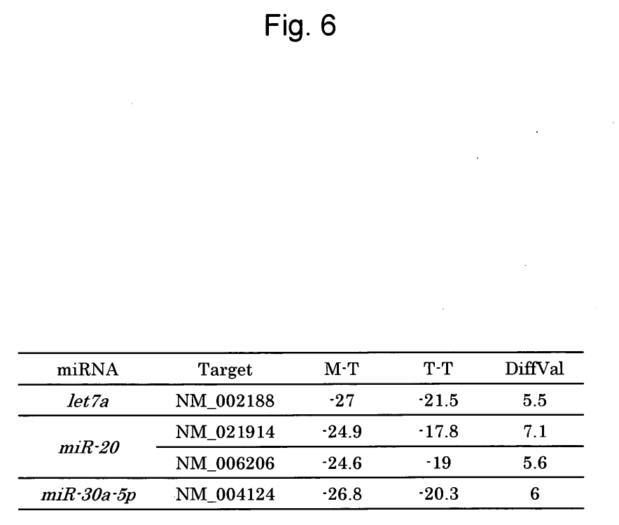

From the results obtained by Example 1, a total of four types of sets of human miRNAs and target mRNAs thereof, namely sets of let7a/NM_{—}002188, miR-20/NM_{—}021914, miR-20/NM_{—}006206, and miR-30a-5p/NM_{—}004124, was selected (FIG. 5). Then, the translation inhibitory activity of the miRNA on each target mRNA was examined by experiments. Concerning the selected four types of sets of miRNAs and target mRNAs, the secondary structure energy between miRNA and MRE (M-T), the most stable secondary structure energy which can be formed by MRE with the partial sequence within the mRNA (T-T), and the difference between “M-T” and “T-T” (DiffVal) are shown in FIG. 6.

### (1) Dual Luciferase Assay

In order to readily detect the translation inhibitory activity concerning the selected four types of sets of miRNAs and target mRNAs, one copy of each estimated MRE was inserted into the immediate downstream of the luciferase-coding region (XbaI/NotI site) on a plasmid pRL-TK. The protein-translation inhibitory activity of the endogenous miRNA was examined by observing the influences of the MRE insertion on the expression of this marker protein.

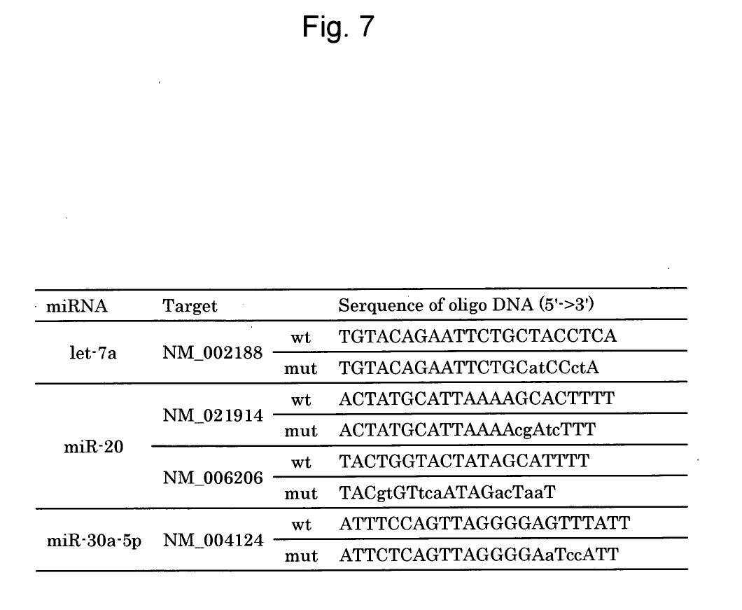

Human HeLa S3 (SC) cells were cultured in Dulbecco's modified Eagle's medium (DMEM) containing 10% fetal bovine serum (FBS). The HeLa S3 (SC) cells were confirmed to express let7a, miR-20, and miR-30a-5p by Northern blot analysis using small molecule-RNA fractions adjusted with mirVana miRNA isolation kit (Ambion). Oligo DNAs having the MRE sequence of each target mRNA (wild type) and a mutated MRE sequence thereof (mutant type) were respectively synthesized (FIG. 7), which were inserted into the downstream of Aequorea victoria luciferase-coding region (XbaI/NotI site) on a plasmid pRL-TK. Adhered HeLa S3 (SC) cells were cultured in the 10% FBS-containing DMEM medium until 50% to 80% confluent in a 96-well culture dish or a 24-well culture dish. In the 96-well culture dish, for the purpose of normalization, in addition to 100 ng of each of the above recombinant plasmid, 315 ng of a plasmid pGL which expresses a fixed amount of firefly luciferase were transfected altogether into human HeLa S3 (SC) cells using the CalPhos mammalian transfection kit (Clontech).

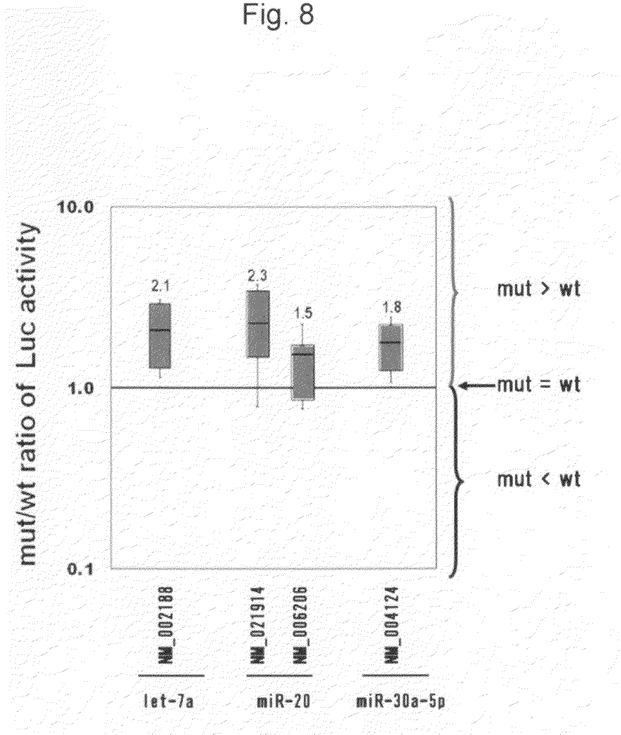

After 20 to 24 hours, a lysate of the transfected cells was produced, and two types of luciferase activities were assayed using Dual Luciferase Reporter Assay System (Promega). Values of the Aequorea victoria luciferase activity were normalized by values of the firefly luciferase activity. The normalized Aequorea victoria luciferase activities between the wild type and the mutant type were compared. As a result, the activity of the mutant type was higher than that of the wild type regarding each MRE (FIG. 8). This shows that a corresponding endogenous miRNA interacts with MRE to suppress the translational process in the target mRNA.

### (2) GFP Reporter Assay

HeLa S3 (SC) cells were cultured in a 12-well culture dish in the same manner as that of the above luciferase assay. Next, synthesized DNAs (⁵′gggatccACCGGATAATCTAGAGCGGCCGCT³′ and ⁵′GATCAGCGGCCGCTCTAGATTATCCGGTGGATCCC³′) were annealed into the SmaI/BclI site of a pEGFP-C1 vector (Clontech) to effect a modification to form the XbaI/NotI site after the stop codon of the pEGFP-C1 vector (Clontech). The modified pEGFP-C1 vector was named pEGFP-C1-NotI. Concerning sets of let-7a/NM_{—}002188 and miR-20/NM_{—}021914, one copy of each of the above two types of MREs was inserted into the XbaI/NotI site on the downstream of the green fluorescent protein (GFP)-coding region on the plasmid pEGFP-C1-NotI. The resultant products were transfected into human HeLa S3 (SC) cells using the CalPhos mammalian transfection kit (Clontech). After 24 hours from the transfection, proteins and RNAs were prepared from the transfected HeLa S3 (SC) cells in accordance with the protocol using PARIS Protein and RNA Isolation System (Ambion).

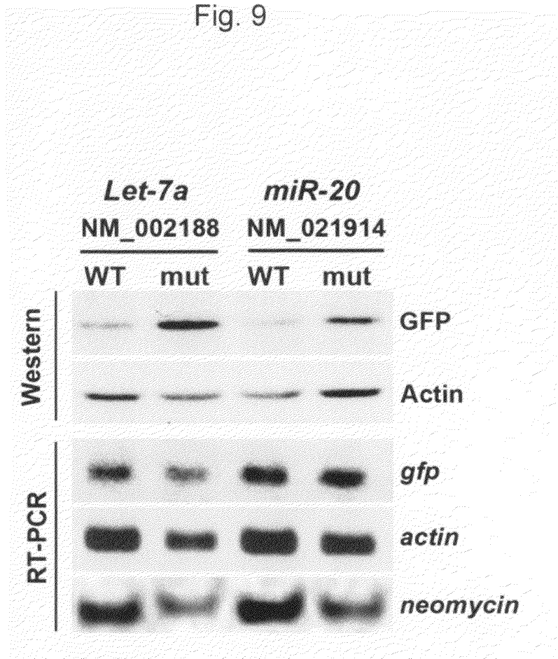

All proteins collected from the human HeLaS3 (SC) cells were subjected to SDS electrophoresis with a 10% polyacrylamide gel, and were transferred onto a PVDF membrane through electroblotting. The membrane was treated with a rabbit anti-GFP polyclonal antibody (BD living Colors A.V. peptide antibody (Clontech)) and a rabbit anti-actin antibody, and was further treated with an anti-rabbit immunoglobulin G antibody conjugated to horseradish peroxidase. The peroxidase activity of the immunocomplex was visualized by an ELC Detection Kit (Amersham) and analyzed by the LAS-1000 plus lumino-image analyzer (Fuji Film) (FIG. 9).

Moreover, all mRNAs collected from the human HeLa S3 (SC) cells were treated with Dnase I and amplified as 2-4 ng, 4 ng, or 8 ng templates using the SuperScript One-Step RT-PCR with Platinum Taq kit (Invitrogen), respectively concerning GFP, b-Actin, or Neomycin. The following primers were used.

### 1) Primers

### 1a) EGFP Primer:

| | |
|---|---|
| Forward: | 5′ACTACCTGAGCACCCAGTCCG |
| Reverse: | 5′CGGACTTCTACAGCTCGTCCAT |

Size of amplified fragment (Amplicon size): 123 bp

### 1b) Neomycin Primer:

| | |
|---|---|
| Forward: | 5′GACCGCTATCAGGACATAGCGTT |
| Reverse: | 5′AAGAACTCGCAAGAAGGCGATAGA. |

Amplicon size: 144 bp

### 1c) Actin Primer:

| | |
|---|---|
| Forward: | 5′GCTCACCATGGATGATGATATCGC |
| Reverse: | 5′GACCTGGCCGTCAGGCAGCTCG |

Amplicon size: 748 bp

The reverse transcription was performed at 55° C. for 20 minutes. The PCR amplification was performed by 25 cycles (30 sec at 95° C., 30 sec at 60° C., and 1 min at 72° C.). The PCR products were separated on an agarose gel or a polyacrylamide gel, stained with ethidium bromide or cyber green, and analyzed by the LAS-1000 plus lumino-image analyzer (Fuji Film).

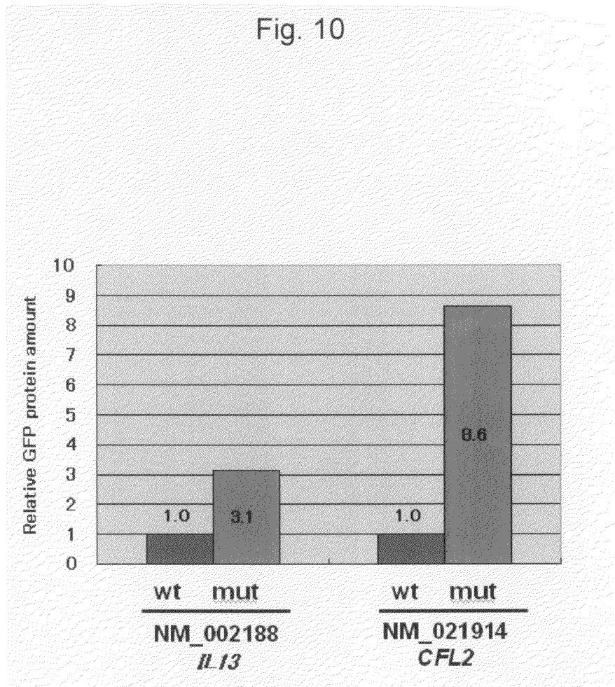

Regarding cells transfected with the plasmid (wt) having the wild type MRE inserted therein and cells transfected with the plasmid (mut) having the mutant type MRE inserted therein, the quantitative RT-PCR results show that GFP- and Actin-encoding mRNAs, gfp, and actin were all equivalently transcribed, whereas the Western blot results show that the Actin protein expression level was equivalent but the GFP protein expression level was apparently higher in mut. The quantification of the results of FIG. 9 show that the mutant type of the set of let-7a/NM_{—}002188 was 3.1 times greater in the expression level than the wild type, and the mutant type of the set of miR-20/NM_{—}021914 was 8.6 times greater in the expression level than the wild type (FIG. 10). That is to say, it was confirmed that the GFP expression was suppressed by let-7a and miR-20 under the presence of MRE, and further that the expression was suppressed not in the transcriptional stage but in the translational stage.

From the above, it was confirmed that the protein translation was controlled in miRNAs and target mRNAs estimated by the method of the present invention, and at the same time the protein expression was shown to be controllable. These results also show that the present invention is effective for the development of the treatment for diseases associated with the above proteins and the treatment per se for diseases.

### INDUSTRIAL APPLICABILITY

According to the present invention, with a combination of sets of miRNAs and target mRNAs estimated by the method for searching for target mRNAs of functional RNA of the present invention, the expression of proteins encoded by the target mRNAs can be controlled, and the method can be utilized in the technical fields of genetic engineering.

Further, according to the present invention, expressions of proteins such as an Interleukin 13, a Cofilin 2 variant 1, a Platelet-derived growth factor receptor, alpha polypeptide, and a Glia maturation factor, beta can be controlled by let-7a, miR-20, and miR-30a-5p.

Moreover, the development of treatments for diseases and treatments for diseases become possible with use of protein expression control of the present invention.

All partial sequences, publications, patents, and patent applications cited herein are incorporated herein by reference in their entirety.

1. A method of predicting or identifying target mRNA(s) the expression of which is controlled by 16 to 25 bases-long miRNA molecule(s) having a gene expression regulatory function, comprising:
(1) a first step of serially calculating secondary structure energies between partial sequences in target mRNA candidates and miRNA sequence(s) to search for stably-bindable partial sequences with the miRNAs, so as to select sets of miRNAs and target mRNA candidates having such stably-bindable partial sequences; and
(2) a second step of calculating, among sets of miRNAs and target mRNA candidates selected in the first step, the most stable secondary structure energies, which can be formed by said partial sequences that are stably-bindable with the miRNAs or sequences in the vicinity thereof within the mRNA, among target mRNA candidates, and comparing them with secondary structure energies between miRNA sequences and said stably-bindable partial sequences in target mRNA candidates, so as to select set(s) of miRNA and mRNA having said partial sequence or a sequence in the vicinity thereof such that the most stable secondary energy which can be formed by said partial sequence or the sequence in the vicinity thereof within said mRNA, is relatively high,
wherein the expression of the mRNA in the set selected by the above steps is predicted or identified to be regulated by the miRNA molecule in the concerned set.

2. A method of predicting or identifying target mRNA(s) the expression of which is controlled by 16 to 25 bases-long miRNA molecule(s) having a gene expression regulatory function, comprising:
(1) a first step of, concerning a certain organism species, serially calculating secondary structure energies between partial sequences in target mRNA candidates and miRNA sequence(s) to search for stably-bindable partial sequences with the miRNAs, so as to select sets of miRNAs and target mRNA candidates having such stably-bindable partial sequences; and
(2) a second step of calculating, among sets of miRNAs and target mRNA candidates selected in the first step, the most stable secondary structure energies, which can be formed by said partial sequences that are stably-bindable with the miRNAs or sequences in the vicinity thereof within the mRNA, among target mRNA candidates, and comparing them with secondary structure energies between miRNA sequences and said stably-bindable partial sequences in target mRNA candidates, so as to select set(s) of miRNA and mRNA having said partial sequence or a sequence in the vicinity thereof such that the most stable secondary energy which can be formed by said partial sequence or the sequences in the vicinity thereof within said mRNA, is relatively high,
(3) a step of performing the first step and the second step concerning a different organism species so as to select set(s) of miRNAs and mRNAs having binding environments that are formed by miRNAs and partial structures of mRNA sequences preserved among these organism species,
wherein the expression of the mRNA in the set selected by the above steps is predicted or identified to be regulated by the miRNA molecule in the concerned set.

3. The method according to either one of claims 1 or 2, wherein a length of a partial sequence of mRNA is elongated by 3 to 8 base pairs as compared to a length of miRNA in the first step.

4. The method according to claim 3, wherein a partial sequence is searched by shifting serially by one base from the 3′-end of a target mRNA candidate.

5. The method according to either one of claims 1 or 2, wherein a partial sequence is searched from a 3′-UTR region of a target mRNA candidate in the first step.

6. The method according to either one of claims 1 or 2, wherein the calculation of binding energy is speeded-up by considering only Watson-Click base pairs, G-U wobble base pairs, bulge loops, and internal loops with use of an approach of RNA secondary structure prediction, in the first step.

7. The method according to either one of claims 1 or 2, wherein said second step uses an approach of assuming the partial sequences in the vicinity to be 0 to 20 bases-shifted positions from a partial sequence selected in the first step, for calculating c the most stable secondary energy which can be formed thereby within mRNA.

8. The method of predicting or identifying target mRNA(s) the expression of which is controlled by miRNA molecule(s) according to either one of claims 1 or 2, further comprising a step of transfecting an miRNA molecule into a cell and confirming an influence on the expression of the target mRNA.

9. The method of predicting or identifying target mRNA(s) the expression of which is controlled by miRNA molecule(s) according to either one of claims 1 or 2, further comprising examining an influence on expressions of target mRNA candidates by detecting target mRNA candidates or corresponding cDNAs, using a DNA/RNA chip on the surface of which a plurality of target mRNA candidates or complementary strands thereof are arranged.

10. The method of predicting or identifying target mRNA(s) controlled by miRNA molecule(s) according to either one of claims 1 or 2, wherein the miRNA(s) are represented by any one of SEQ IDs: 1 to 238.

11. An Xn gene expression regulatory agent, respectively comprising a nucleic acid represented by Yn as an active ingredient for regulating the expression of the Xn gene which represents as follows (wherein n=1, 2, 3, or 4),
X1: Interleukin 13 (NM_{—}002188),
X2: Cofilin 2 variant 1 (NM_{—}021914),
X3: Platelet-derived growth factor receptor, alpha polypeptide (NM_{—}006206),
X4: Glia maturation factor, beta (NM_{—}004124),
Y1: (1) UGAGGUAGUAGGUUGUAUAGUU,
Y2: (2) UAAAGUGCUUAUAGUGCAGGUA,
Y3: (3) UAAAGUGCUUAUAGUGCAGGUA, and
Y4: (3) UGUAAACAUCCUCGACUGGAAGC.

12. A medicament comprising the Xn gene expression regulatory agent according to claim 11 as an active ingredient (wherein: n=1, 2, 3, or 4; X1 represents an Interleukin 13 (NM_{—}002188); X2 represents a Cofilin 2 variant 1 (NM_{—}021914); X3 represents a platelet-derived growth factor receptor, alpha polypeptide (NM_{—}006206); and X4 represents a Glia maturation factor, beta (NM_{—}004124)).

13. A method of controlling the expression of a target mRNA with use of an miRNA molecule, wherein the expression of the target mRNA has been estimated or identified to be controlled with use of the miRNA molecule by a method according to either one of claims 1 or 2.

14. A method of controlling a biofunction (of a non-human organism) by controlling the expression of a target gene (target mRNA) with use of an miRNA, wherein the expression of the target gene (target mRNA) is estimated or identified with use of the miRNA molecule by a method according to either one of claims 1 or 2.

15. The method of controlling a biofunction (of a non-human organism) according to claim 14, for the development of the treatment of a disease.

16. The method of controlling a biofunction (of a non-human organism) according to claim 14, for the treatment of a disease.


SEQUENCE LISTING (258 sequences)

SEQ ID NO: 1 - RNA, 22 nt, Homo sapiens
ugagguaguagguuguauaguu

SEQ ID NO: 2 - RNA, 22 nt, Homo sapiens
ugagguaguagguugugugguu

SEQ ID NO: 3 - RNA, 22 nt, Homo sapiens
ugagguaguagguuguaugguu

SEQ ID NO: 4 - RNA, 21 nt, Homo sapiens
agagguaguagguugcauagu

SEQ ID NO: 5 - RNA, 21 nt, Homo sapiens
ugagguaggagguuguauagu

SEQ ID NO: 6 - RNA, 22 nt, Homo sapiens
ugagguaguagauuguauaguu

SEQ ID NO: 7 - RNA, 21 nt, Homo sapiens
ugagguaguaguuuguacagu

SEQ ID NO: 8 - RNA, 19 nt, Homo sapiens
ugagguaguaguuugugcu

SEQ ID NO: 9 - RNA, 21 nt, Homo sapiens
uggaauguaaagaaguaugua

SEQ ID NO: 10 - RNA, 22 nt, Homo sapiens
uacaguacugugauaacugaag

SEQ ID NO: 11 - RNA, 23 nt, Homo sapiens
agcagcauuguacagggcuauga

SEQ ID NO: 12 - RNA, 24 nt, Homo sapiens
aaaagugcuuacagugcagguagc

SEQ ID NO: 13 - RNA, 21 nt, Homo sapiens
uaaagugcugacagugcagau

SEQ ID NO: 14 - RNA, 23 nt, Homo sapiens
agcagcauuguacagggcuauca

SEQ ID NO: 15 - RNA, 23 nt, Homo sapiens
uggagugugacaaugguguuugu

SEQ ID NO: 16 - RNA, 22 nt, Homo sapiens
uuaaggcacgcggugaaugcca

SEQ ID NO: 17 - RNA, 21 nt, Homo sapiens
ucguaccgugaguaauaaugc

SEQ ID NO: 18 - RNA, 22 nt, Homo sapiens
ucggauccgucugagcuuggcu

SEQ ID NO: 19 - RNA, 22 nt, Homo sapiens
ucacagugaaccggucucuuuu

SEQ ID NO: 20 - RNA, 22 nt, Homo sapiens
ucacagugaaccggucucuuuc

SEQ ID NO: 21 - RNA, 21 nt, Homo sapiens
cuuuuugcggucugggcuugc

SEQ ID NO: 22 - RNA, 20 nt, Homo sapiens
cagugcaauguuaaaagggc

SEQ ID NO: 23 - RNA, 22 nt, Homo sapiens
cagugcaaugaugaaagggcau

SEQ ID NO: 24 - RNA, 22 nt, Homo sapiens
uaacagucuacagccauggucg

SEQ ID NO: 25 - RNA, 22 nt, Homo sapiens
uugguccccuucaaccagcugu

SEQ ID NO: 26 - RNA, 21 nt, Homo sapiens
uugguccccuucaaccagcua

SEQ ID NO: 27 - RNA, 21 nt, Homo sapiens
ugugacugguugaccagaggg

SEQ ID NO: 28 - RNA, 23 nt, Homo sapiens
uauggcuuuuuauuccuauguga

SEQ ID NO: 29 - RNA, 22 nt, Homo sapiens
uauggcuuuucauuccuaugug

SEQ ID NO: 30 - RNA, 23 nt, Homo sapiens
acuccauuuguuuugaugaugga

SEQ ID NO: 31 - RNA, 22 nt, Homo sapiens
uauugcuuaagaauacgcguag

SEQ ID NO: 32 - RNA, 17 nt, Homo sapiens
agcugguguugugaauc

SEQ ID NO: 33 - RNA, 18 nt, Homo sapiens
ucuacagugcacgugucu

SEQ ID NO: 34 - RNA, 21 nt, Homo sapiens
agugguuuuacccuaugguag

SEQ ID NO: 35 - RNA, 21 nt, Homo sapiens
aacacugucugguaaagaugg

SEQ ID NO: 36 - RNA, 23 nt, Homo sapiens
uguaguguuuccuacuuuaugga

SEQ ID NO: 37 - RNA, 22 nt, Homo sapiens
ugagaugaagcacuguagcuca

SEQ ID NO: 38 - RNA, 22 nt, Homo sapiens
uacaguauagaugauguacuag

SEQ ID NO: 39 - RNA, 22 nt, Homo sapiens
ugagaacugaauuccauggguu

SEQ ID NO: 40 - RNA, 22 nt, Homo sapiens
ucuggcuccgugucuucacucc

SEQ ID NO: 41 - RNA, 22 nt, Homo sapiens
acuagacugaagcuccuugagg

SEQ ID NO: 42 - RNA, 21 nt, Homo sapiens
ucagugcaugacagaacuugg

SEQ ID NO: 43 - RNA, 20 nt, Homo sapiens
uugcauagucacaaaaguga

SEQ ID NO: 44 - RNA, 22 nt, Homo sapiens
uagguuauccguguugccuucg

SEQ ID NO: 45 - RNA, 22 nt, Homo sapiens
uuaaugcuaaucgugauagggg

SEQ ID NO: 46 - RNA, 22 nt, Homo sapiens
uagcagcacauaaugguuugug

SEQ ID NO: 47 - RNA, 22 nt, Homo sapiens
uagcagcacaucaugguuuaca

SEQ ID NO: 48 - RNA, 20 nt, Homo sapiens
acugcagugaaggcacuugu

SEQ ID NO: 49 - RNA, 24 nt, Homo sapiens
caaagugcuuacagugcagguagu

SEQ ID NO: 50 - RNA, 22 nt, Homo sapiens
uaaggugcaucuagugcagaua

SEQ ID NO: 51 - RNA, 23 nt, Homo sapiens
aacauucaacgcugucggugagu

SEQ ID NO: 52 - RNA, 22 nt, Homo sapiens
aacauucaaccugucggugagu

SEQ ID NO: 53 - RNA, 22 nt, Homo sapiens
uuuggcaaugguagaacucaca

SEQ ID NO: 54 - RNA, 23 nt, Homo sapiens
uauggcacugguagaauucacug

SEQ ID NO: 55 - RNA, 22 nt, Homo sapiens
uggacggagaacugauaagggu

SEQ ID NO: 56 - RNA, 18 nt, Homo sapiens
uggagagaaaggcaguuc

SEQ ID NO: 57 - RNA, 21 nt, Homo sapiens
ucgugucuuguguugcagccg

SEQ ID NO: 58 - RNA, 22 nt, Homo sapiens
caucccuugcaugguggagggu

SEQ ID NO: 59 - RNA, 23 nt, Homo sapiens
gugccuacugagcugauaucagu

SEQ ID NO: 60 - RNA, 22 nt, Homo sapiens
ugauauguuugauauauuaggu

SEQ ID NO: 61 - RNA, 21 nt, Homo sapiens
aacuggccuacaaagucccag

SEQ ID NO: 62 - RNA, 21 nt, Homo sapiens
uagguaguuucauguuguugg

SEQ ID NO: 63 - RNA, 21 nt, Homo sapiens
uagguaguuuccuguuguugg

SEQ ID NO: 64 - RNA, 23 nt, Homo sapiens
cccaguguucagacuaccuguuc

SEQ ID NO: 65 - RNA, 23 nt, Homo sapiens
cccaguguuuagacuaucuguuc

SEQ ID NO: 66 - RNA, 23 nt, Homo sapiens
ugugcaaaucuaugcaaaacuga

SEQ ID NO: 67 - RNA, 22 nt, Homo sapiens
uaaagugcuuauagugcaggua

SEQ ID NO: 68 - RNA, 22 nt, Homo sapiens
aauacugccggguaaugaugga

SEQ ID NO: 69 - RNA, 22 nt, Homo sapiens
uccuucauuccaccggagucug

SEQ ID NO: 70 - RNA, 22 nt, Homo sapiens
uggaauguaaggaagugugugg

SEQ ID NO: 71 - RNA, 22 nt, Homo sapiens
uagcuuaucagacugauguuga

SEQ ID NO: 72 - RNA, 21 nt, Homo sapiens
cugugcgugugacagcggcug

SEQ ID NO: 73 - RNA, 22 nt, Homo sapiens
uucccuuugucauccuucgccu

SEQ ID NO: 74 - RNA, 21 nt, Homo sapiens
uaacagucuccagucacggcc

SEQ ID NO: 75 - RNA, 21 nt, Homo sapiens
acagcaggcacagacaggcag

SEQ ID NO: 76 - RNA, 21 nt, Homo sapiens
uaaucucagcuggcaacugug

SEQ ID NO: 77 - RNA, 21 nt, Homo sapiens
uugugcuugaucuaaccaugu

SEQ ID NO: 78 - RNA, 22 nt, Homo sapiens
aagcugccaguugaagaacugu

SEQ ID NO: 79 - RNA, 23 nt, Homo sapiens
agcuacauugucugcuggguuuc

SEQ ID NO: 80 - RNA, 24 nt, Homo sapiens
agcuacaucuggcuacugggucuc

SEQ ID NO: 81 - RNA, 21 nt, Homo sapiens
ugucaguuugucaaauacccc

SEQ ID NO: 82 - RNA, 21 nt, Homo sapiens
aucacauugccagggauuucc

SEQ ID NO: 83 - RNA, 23 nt, Homo sapiens
aucacauugccagggauuaccac

SEQ ID NO: 84 - RNA, 22 nt, Homo sapiens
uggcucaguucagcaggaacag

SEQ ID NO: 85 - RNA, 22 nt, Homo sapiens
cauugcacuugucucggucuga

SEQ ID NO: 86 - RNA, 22 nt, Homo sapiens
uucaaguaauccaggauaggcu

SEQ ID NO: 87 - RNA, 22 nt, Homo sapiens
uucacaguggcuaaguuccgcc

SEQ ID NO: 88 - RNA, 20 nt, Homo sapiens
uucacaguggcuaaguucug

SEQ ID NO: 89 - RNA, 22 nt, Homo sapiens
aaggagcucacagucuauugag

SEQ ID NO: 90 - RNA, 21 nt, Homo sapiens
agggcccccccucaauccugu

SEQ ID NO: 91 - RNA, 22 nt, Homo sapiens
uagcaccauuugaaaucgguua

SEQ ID NO: 92 - RNA, 22 nt, Homo sapiens
cuuucagucggauguuugcagc

SEQ ID NO: 93 - RNA, 23 nt, Homo sapiens
uguaaacauccucgacuggaagc

SEQ ID NO: 94 - RNA, 21 nt, Homo sapiens
uguaaacauccuacacucagc

SEQ ID NO: 95 - RNA, 22 nt, Homo sapiens
uguaaacauccccgacuggaag

SEQ ID NO: 96 - RNA, 20 nt, Homo sapiens
uguaaacauccuugacugga

SEQ ID NO: 97 - RNA, 21 nt, Homo sapiens
ggcaagaugcuggcauagcug

SEQ ID NO: 98 - RNA, 23 nt, Homo sapiens
aaaagcuggguugagagggcgaa

SEQ ID NO: 99 - RNA, 22 nt, Homo sapiens
gcacauuacacggucgaccucu

SEQ ID NO: 100 - RNA, 22 nt, Homo sapiens
ccacugccccaggugcugcugg

SEQ ID NO: 101 - RNA, 23 nt, Homo sapiens
cgcauccccuagggcauuggugu

SEQ ID NO: 102 - RNA, 21 nt, Homo sapiens
ccuaguagguguccaguaagu

SEQ ID NO: 103 - RNA, 20 nt, Homo sapiens
ccucugggcccuuccuccag

SEQ ID NO: 104 - RNA, 22 nt, Homo sapiens
cuggcccucucugcccuuccgu

SEQ ID NO: 105 - RNA, 19 nt, Homo sapiens
gugcauuguaguugcauug

SEQ ID NO: 106 - RNA, 23 nt, Homo sapiens
gcaaagcacacggccugcagaga

SEQ ID NO: 107 - RNA, 21 nt, Homo sapiens
gccccugggccuauccuagaa

SEQ ID NO: 108 - RNA, 23 nt, Homo sapiens
ucaagagcaauaacgaaaaaugu

SEQ ID NO: 109 - RNA, 23 nt, Homo sapiens
uccagcaucagugauuuuguuga

SEQ ID NO: 110 - RNA, 21 nt, Homo sapiens
ucccuguccuccaggagcuca

SEQ ID NO: 111 - RNA, 21 nt, Homo sapiens
ugcugacuccuaguccagggc

SEQ ID NO: 112 - RNA, 23 nt, Homo sapiens
ugucugcccgcaugccugccucu

SEQ ID NO: 113 - RNA, 22 nt, Homo sapiens
uggcagugucuuagcugguugu

SEQ ID NO: 114 - RNA, 22 nt, Homo sapiens
aggcagugucauuagcugauug

SEQ ID NO: 115 - RNA, 22 nt, Homo sapiens
aggcaguguaguuagcugauug

SEQ ID NO: 116 - RNA, 23 nt, Homo sapiens
ucuuugguuaucuagcuguauga

SEQ ID NO: 117 - RNA, 22 nt, Homo sapiens
uauugcacuugucccggccugu

SEQ ID NO: 118 - RNA, 22 nt, Homo sapiens
aaagugcuguucgugcagguag

SEQ ID NO: 119 - RNA, 22 nt, Homo sapiens
ugagguaguaaguuguauuguu

SEQ ID NO: 120 - RNA, 22 nt, Mus musculus
ugagguaguagguuguauaguu

SEQ ID NO: 121 - RNA, 22 nt, Mus musculus
ugagguaguagguugugugguu

SEQ ID NO: 122 - RNA, 22 nt, Mus musculus
ugagguaguagguuguaugguu

SEQ ID NO: 123 - RNA, 21 nt, Mus musculus
agagguaguagguugcauagu

SEQ ID NO: 124 - RNA, 21 nt, Mus musculus
ugagguaggagguuguauagu

SEQ ID NO: 125 - RNA, 22 nt, Mus musculus
ugagguaguagauuguauaguu

SEQ ID NO: 126 - RNA, 21 nt, Mus musculus
ugagguaguaguuuguacagu

SEQ ID NO: 127 - RNA, 19 nt, Mus musculus
ugagguaguaguuugugcu

SEQ ID NO: 128 - RNA, 21 nt, Mus musculus
uggaauguaaagaaguaugua

SEQ ID NO: 129 - RNA, 20 nt, Mus musculus
uacaguacugugauaacuga

SEQ ID NO: 130 - RNA, 23 nt, Mus musculus
agcagcauuguacagggcuauga

SEQ ID NO: 131 - RNA, 22 nt, Mus musculus
caaagugcuaacagugcaggua

SEQ ID NO: 132 - RNA, 21 nt, Mus musculus
uaaagugcugacagugcagau

SEQ ID NO: 133 - RNA, 23 nt, Mus musculus
agcagcauuguacagggcuauca

SEQ ID NO: 134 - RNA, 23 nt, Mus musculus
uggagugugacaaugguguuugu

SEQ ID NO: 135 - RNA, 22 nt, Mus musculus
uuaaggcacgcggugaaugcca

SEQ ID NO: 136 - RNA, 21 nt, Mus musculus
ucguaccgugaguaauaaugc

SEQ ID NO: 137 - RNA, 22 nt, Mus musculus
ucggauccgucugagcuuggcu

SEQ ID NO: 138 - RNA, 22 nt, Mus musculus
ucacagugaaccggucucuuuu

SEQ ID NO: 139 - RNA, 22 nt, Mus musculus
ucacagugaaccggucucuuuc

SEQ ID NO: 140 - RNA, 22 nt, Mus musculus
cuuuuugcggucugggcuugcu

SEQ ID NO: 141 - RNA, 20 nt, Mus musculus
cagugcaauguuaaaagggc

SEQ ID NO: 142 - RNA, 22 nt, Mus musculus
cagugcaaugaugaaagggcau

SEQ ID NO: 143 - RNA, 22 nt, Mus musculus
uaacagucuacagccauggucg

SEQ ID NO: 144 - RNA, 22 nt, Mus musculus
uugguccccuucaaccagcugu

SEQ ID NO: 145 - RNA, 21 nt, Mus musculus
uugguccccuucaaccagcua

SEQ ID NO: 146 - RNA, 21 nt, Mus musculus
ugugacugguugaccagaggg

SEQ ID NO: 147 - RNA, 23 nt, Mus musculus
uauggcuuuuuauuccuauguga

SEQ ID NO: 148 - RNA, 22 nt, Mus musculus
uauggcuuuucauuccuaugug

SEQ ID NO: 149 - RNA, 23 nt, Mus musculus
acuccauuuguuuugaugaugga

SEQ ID NO: 150 - RNA, 22 nt, Mus musculus
uauugcuuaagaauacgcguag

SEQ ID NO: 151 - RNA, 17 nt, Mus musculus
agcugguguugugaauc

SEQ ID NO: 152 - RNA, 18 nt, Mus musculus
ucuacagugcacgugucu

SEQ ID NO: 153 - RNA, 21 nt, Mus musculus
agugguuuuacccuaugguag

SEQ ID NO: 154 - RNA, 21 nt, Mus musculus
aacacugucugguaaagaugg

SEQ ID NO: 155 - RNA, 22 nt, Mus musculus
uguaguguuuccuacuuuaugg

SEQ ID NO: 156 - RNA, 22 nt, Mus musculus
ugagaugaagcacuguagcuca

SEQ ID NO: 157 - RNA, 22 nt, Mus musculus
uacaguauagaugauguacuag

SEQ ID NO: 158 - RNA, 22 nt, Mus musculus
ugagaacugaauuccauggguu

SEQ ID NO: 159 - RNA, 22 nt, Mus musculus
ucuggcuccgugucuucacucc

SEQ ID NO: 160 - RNA, 21 nt, Mus musculus
cuagacugaggcuccuugagg

SEQ ID NO: 161 - RNA, 21 nt, Mus musculus
ucagugcaugacagaacuugg

SEQ ID NO: 162 - RNA, 20 nt, Mus musculus
uugcauagucacaaaaguga

SEQ ID NO: 163 - RNA, 22 nt, Mus musculus
uagguuauccguguugccuucg

SEQ ID NO: 164 - RNA, 22 nt, Mus musculus
uuaaugcuaauugugauagggg

SEQ ID NO: 165 - RNA, 22 nt, Mus musculus
uagcagcacauaaugguuugug

SEQ ID NO: 166 - RNA, 22 nt, Mus musculus
uagcagcacaucaugguuuaca

SEQ ID NO: 167 - RNA, 20 nt, Mus musculus
acugcagugagggcacuugu

SEQ ID NO: 168 - RNA, 24 nt, Mus musculus
caaagugcuuacagugcagguagu

SEQ ID NO: 169 - RNA, 22 nt, Mus musculus
uaaggugcaucuagugcagaua

SEQ ID NO: 170 - RNA, 23 nt, Mus musculus
aacauucaacgcugucggugagu

SEQ ID NO: 171 - RNA, 22 nt, Mus musculus
aacauucaaccugucggugagu

SEQ ID NO: 172 - RNA, 22 nt, Mus musculus
uuuggcaaugguagaacucaca

SEQ ID NO: 173 - RNA, 23 nt, Mus musculus
uauggcacugguagaauucacug

SEQ ID NO: 174 - RNA, 22 nt, Mus musculus
uggacggagaacugauaagggu

SEQ ID NO: 175 - RNA, 18 nt, Mus musculus
uggagagaaaggcaguuc

SEQ ID NO: 176 - RNA, 22 nt, Mus musculus
ucgugucuuguguugcagccgg

SEQ ID NO: 177 - RNA, 22 nt, Mus musculus
caucccuugcaugguggagggu

SEQ ID NO: 178 - RNA, 23 nt, Mus musculus
gugccuacugagcugauaucagu

SEQ ID NO: 179 - RNA, 22 nt, Mus musculus
ugauauguuugauauauuaggu

SEQ ID NO: 180 - RNA, 21 nt, Mus musculus
aacuggccuacaaagucccag

SEQ ID NO: 181 - RNA, 21 nt, Mus musculus
uagguaguuucauguuguugg

SEQ ID NO: 182 - RNA, 21 nt, Mus musculus
uagguaguuuccuguuguugg

SEQ ID NO: 183 - RNA, 23 nt, Mus musculus
cccaguguucagacuaccuguuc

SEQ ID NO: 184 - RNA, 23 nt, Mus musculus
cccaguguuuagacuaccuguuc

SEQ ID NO: 185 - RNA, 23 nt, Mus musculus
ugugcaaaucuaugcaaaacuga

SEQ ID NO: 186 - RNA, 23 nt, Mus musculus
uaaagugcuuauagugcagguag

SEQ ID NO: 187 - RNA, 22 nt, Mus musculus
aauacugccggguaaugaugga

SEQ ID NO: 188 - RNA, 22 nt, Mus musculus
uccuucauuccaccggagucug

SEQ ID NO: 189 - RNA, 22 nt, Mus musculus
uggaauguaaggaagugugugg

SEQ ID NO: 190 - RNA, 22 nt, Mus musculus
uagcuuaucagacugauguuga

SEQ ID NO: 191 - RNA, 21 nt, Mus musculus
cugugcgugugacagcggcug

SEQ ID NO: 192 - RNA, 22 nt, Mus musculus
uucccuuugucauccuuugccu

SEQ ID NO: 193 - RNA, 21 nt, Mus musculus
uaacagucuccagucacggcc

SEQ ID NO: 194 - RNA, 21 nt, Mus musculus
acagcaggcacagacaggcag

SEQ ID NO: 195 - RNA, 21 nt, Mus musculus
uaaucucagcuggcaacugug

SEQ ID NO: 196 - RNA, 21 nt, Mus musculus
uugugcuugaucuaaccaugu

SEQ ID NO: 197 - RNA, 22 nt, Mus musculus
aagcugccaguugaagaacugu

SEQ ID NO: 198 - RNA, 22 nt, Mus musculus
agcuacauugucugcuggguuu

SEQ ID NO: 199 - RNA, 23 nt, Mus musculus
agcuacaucuggcuacugggucu

SEQ ID NO: 200 - RNA, 21 nt, Mus musculus
ugucaguuugucaaauacccc

SEQ ID NO: 201 - RNA, 21 nt, Mus musculus
aucacauugccagggauuucc

SEQ ID NO: 202 - RNA, 23 nt, Mus musculus
aucacauugccagggauuaccac

SEQ ID NO: 203 - RNA, 22 nt, Mus musculus
uggcucaguucagcaggaacag

SEQ ID NO: 204 - RNA, 22 nt, Mus musculus
cauugcacuugucucggucuga

SEQ ID NO: 205 - RNA, 22 nt, Mus musculus
uucaaguaauccaggauaggcu

SEQ ID NO: 206 - RNA, 21 nt, Mus musculus
uucacaguggcuaaguuccgc

SEQ ID NO: 207 - RNA, 20 nt, Mus musculus
uucacaguggcuaaguucug

SEQ ID NO: 208 - RNA, 22 nt, Mus musculus
aaggagcucacagucuauugag

SEQ ID NO: 209 - RNA, 21 nt, Mus musculus
agggcccccccucaauccugu

SEQ ID NO: 210 - RNA, 22 nt, Mus musculus
uagcaccauuugaaaucgguua

SEQ ID NO: 211 - RNA, 22 nt, Mus musculus
cuuucagucggauguuugcagc

SEQ ID NO: 212 - RNA, 23 nt, Mus musculus
uguaaacauccucgacuggaagc

SEQ ID NO: 213 - RNA, 21 nt, Mus musculus
uguaaacauccuacacucagc

SEQ ID NO: 214 - RNA, 22 nt, Mus musculus
uguaaacauccccgacuggaag

SEQ ID NO: 215 - RNA, 20 nt, Mus musculus
uguaaacauccuugacugga

SEQ ID NO: 216 - RNA, 22 nt, Mus musculus
aggcaagaugcuggcauagcug

SEQ ID NO: 217 - RNA, 23 nt, Mus musculus
aaaagcuggguugagagggcgaa

SEQ ID NO: 218 - RNA, 22 nt, Mus musculus
gcacauuacacggucgaccucu

SEQ ID NO: 219 - RNA, 22 nt, Mus musculus
ccacugccccaggugcugcugg

SEQ ID NO: 220 - RNA, 23 nt, Mus musculus
cgcauccccuagggcauuggugu

SEQ ID NO: 221 - RNA, 23 nt, Mus musculus
ccuaguaggugcucaguaagugu

SEQ ID NO: 222 - RNA, 21 nt, Mus musculus
ccucugggcccuuccuccagu

SEQ ID NO: 223 - RNA, 22 nt, Mus musculus
cuggcccucucugcccuuccgu

SEQ ID NO: 224 - RNA, 19 nt, Mus musculus
gugcauuguaguugcauug

SEQ ID NO: 225 - RNA, 23 nt, Mus musculus
gcaaagcacagggccugcagaga

SEQ ID NO: 226 - RNA, 21 nt, Mus musculus
gccccugggccuauccuagaa

SEQ ID NO: 227 - RNA, 23 nt, Mus musculus
ucaagagcaauaacgaaaaaugu

SEQ ID NO: 228 - RNA, 23 nt, Mus musculus
uccagcaucagugauuuuguuga

SEQ ID NO: 229 - RNA, 21 nt, Mus musculus
ucccuguccuccaggagcuca

SEQ ID NO: 230 - RNA, 21 nt, Mus musculus
ugcugaccccuaguccagugc

SEQ ID NO: 231 - RNA, 23 nt, Mus musculus
ugucugcccgagugccugccucu

SEQ ID NO: 232 - RNA, 23 nt, Mus musculus
uggcagugucuuagcugguuguu

SEQ ID NO: 233 - RNA, 23 nt, Mus musculus
uaggcaguguaauuagcugauug

SEQ ID NO: 234 - RNA, 23 nt, Mus musculus
aggcaguguaguuagcugauugc

SEQ ID NO: 235 - RNA, 23 nt, Mus musculus
ucuuugguuaucuagcuguauga

SEQ ID NO: 236 - RNA, 21 nt, Mus musculus
uauugcacuugucccggccug

SEQ ID NO: 237 - RNA, 23 nt, Mus musculus
caaagugcuguucgugcagguag

SEQ ID NO: 238 - RNA, 22 nt, Mus musculus
ugagguaguaaguuguauuguu

SEQ ID NO: 239 - DNA, 22 bp, Artificial Sequence
  Description of Artificial Sequence
tgtacagaattctgctacctca

SEQ ID NO: 240 - DNA, 22 bp, Artificial Sequence
  Description of Artificial Sequence
tgtacagaattctgcatcccta

SEQ ID NO: 241 - DNA, 22 bp, Artificial Sequence
  Description of Artificial Sequence
actatgcattaaaagcactttt

SEQ ID NO: 242 - DNA, 22 bp, Artificial Sequence
  Description of Artificial Sequence
actatgcattaaaacgatcttt

SEQ ID NO: 243 - DNA, 20 bp, Artificial Sequence
  Description of Artificial Sequence
tactggtactatagcatttt

SEQ ID NO: 244 - DNA, 20 bp, Artificial Sequence
  Description of Artificial Sequence
tacgtgttcaatagactaat

SEQ ID NO: 245 - DNA, 23 bp, Artificial Sequence
  Description of Artificial Sequence
atttccagttaggggagtttatt

SEQ ID NO: 246 - DNA, 23 bp, Artificial Sequence
  Description of Artificial Sequence
attctcagttaggggaatccatt

SEQ ID NO: 247 - DNA, 1282 bp, Homo sapiens
aagccacccagcctatgcatccgctcctcaatcctctcctgttggcactgggcctcatggcgcttttgttgaccacggtcattgctctcacttgccttggcggctttgcctccccaggccctgtgcctccctctacagccctcagggagctcattgaggagctggtcaacatcacccagaaccagaaggctccgctctgcaatggcagcatggtatggagcatcaacctgacagctggcatgtactgtgcagccctggaatccctgatcaacgtgtcaggctgcagtgccatcgagaagacccagaggatgctgagcggattctgcccgcacaaggtctcagctgggcagttttccagcttgcatgtccgagacaccaaaatcgaggtggcccagtttgtaaaggacctgctcttacatttaaagaaactttttcgcgagggacagttcaactgaaacttcgaaagcatcattatttgcagagacaggacctgactattgaagttgcagattcatttttctttctgatgtcaaaaatgtcttgggtaggcgggaaggagggttagggaggggtaaaattccttagcttagacctcagcctgtgctgcccgtcttcagcctagccgacctcagccttccccttgcccagggctcagcctggtgggcctcctctgtccagggccctgagctcggtggacccagggatgacatgtccctacacccctcccctgccctagagcacactgtagcattacagtgggtgccccccttgccagacatgtggtgggacagggacccacttcacacacaggcaactgaggcagacagcagctcaggcacacttcttcttggtcttatttattattgtgtgttatttaaatgagtgtgtttgtcaccgttggggattggggaagactgtggctgctagcacttggagccaagggttcagagactcagggccccagcactaaagcagtggacaccaggagtccctggtaataagtactgtgtacagaattctgctacctcactggggtcctggggcctcggagcctcatccgaggcagggtcaggagaggggcagaacagccgctcctgtctgccagccagcagccagctctcagccaacgagtaatttattgtttttccttgtatttaaatattaaatatgttagcaaagagttaatatatagaagggtaccttgaacactgggggaggggacattgaacaagttgtttcattgactatcaaactgaagccagaaataaagttggtgacagat

SEQ ID NO: 248 - DNA, 3093 bp, Homo sapiens
aaacattttttggatgggacaacttgtatttgtccctttcgcttccacgtccaaacccctttaagaaggatgaatgggcaggatgagttagactccttcgctgtatcgtctactgattcttaaaatgtgacaaatctgattggacgacttacatggcttctggagttacagtgaatgatgaagtcatcaaagtttttaatgatatgaaagtaaggaaatcttctacacaagaggagatcaaaaagagaaagaaagcagttctcttctgtttaagcgatgacaaaagacaaataattgtagaggaagcaaagcagatcttggtgggtgacattggtgatactgtagaggacccctacacatcttttgtgaagttgctacctctgaatgattgccgatatgctttgtacgatgccacatacgaaacaaaagagtctaagaaagaagacctagtatttatattctgggctcctgaaagtgcacctttaaaaagcaagatgatttatgctagctctaaagatgccattaaaaagaaatttacaggtattaaacatgagtggcaagtaaatggcttggatgatattaaggaccgttcgacacttggagagaaattgggaggcaatgtagtagtttcacttgaaggaaaaccattataaaatgacagtcaagtgccatctggatcttaaggagcttccatttctccagctcagtccattggaatagtattaggttttggttttttgttgtatttccccctttccactgggcccttccaacacaatgaatgaaggaaatatcatttatttaagcagcctatcagtgattgccattagactgttgaatactgttacttttatatagaacccaaggaatgccttcctgtcatattttagccaaaacaactggttatatgcctcccttgcagcaagcactacaatgtatgtgatcgtcaatgtgaatagcttagaatactgcaaaggataagctaattgaatgccttgaaagtattatccactggtcagatggtcaacttttttcagtattatttatagttggcacttgattgcagttctgtgaggcttgagcattcatacacctcacctgccttggcaagcctattttagtgatatggcagcacggatataacactatgcattaaaagcactttttgtaataagtttaatatcctaaaaggaatgccaattaagttttgttaactgtgtcatcaacttatcctagtacctcagtgttcattcctgttacctgcatatcttcttaaaagaaatagctgttattaatgcctttttgttttccattgagtgtacactactgaataagtgtaggagttttatgtttaccatgtgagtcctgcaacactaaagatattttgaatatcagtcatgatggcaatttctgtataaaagagccttaaatggaacattgttttgagatcaaactccccaccctcacaaaaatggccacgttgcaataaaaattgtggcatattacagaacgttgccttgttttccttggaaattttgcaaaatgttatgtgaaacaacttctagggtaaaaacagctattactaatctctgcactggtcatttgagaattttttttgtacagcattcatgtgtgatattttccagatttgttggatctatttggtttaaaaagtattctatcttaaggccaactaatataaaataccattgttaaagaatggtacttttataaacattagtgtatttatttcctatgtgttaatatgaagatcagaaattattttttgcactttggcataaatacttttcaatatctgatttgttctctggataaattagcatagttatttttttattcacatttacatttctaagtagttgtatagtagaagcaggaagctcttattgcttatttggtcgtaatgaaaataatttgtaaaatgtcctttaaaagtttaatgatacttctgatgtttcggaacagtcatttcacctactatttctgaatatattttgcaaattgaattggaataggaattgatatagcagtcttaaacattagtagtgggatttggctatggtccagactgtgctccttatagagaatttgatctgctcagtgtgagcggtttgctgttagccagggctatttatggcaaacacatgcttttgtatcttgtcatagttatccacaaaggcaaaactggacttgattctactggtatgcaaaacaggcatgctagtaagcagtcagtcgtggctcagaacttaaccccatagctcagaggaatgcttttagcagaaaacaggaaagaaaatatcccttaaaaattttttttgaatgtgtggaagtaattttagtataattagattttttccatatttttgaaagatttttcagatgtgaacattaaaaatagggattaaatgtctaggcttccatttaaaattatatgaatggtttgggatctttttgcactgagcaattttatttcaggcttccagctgtccctgtgagttatcctggacatttcgatggtttttggtaaggccaaactctgataagcaaaacagagaatactgacgtatacttaaccatatgtgtaactgatacttggcaccatggaatttttcattgagttatttcctcattcttttaaaaaataagggactataaatcagttatgtagtatcttttgtttttgtagctgattccttaactttcttgtatgcctctagtaatttcagagattaaatattgctttaaactgtgatactttgatttgctagattgacaaaactgatactaatataattaagttcatctttgaaatacatctttgtgcgtagagccaaaaaaagagataaaattaataatagttcacttgttatttgagattaatttggcatttgaaatgatcattttattttacaatcatttataatgaatcaatgttccagttagctttaaaaggtatacggtgctaattagtaaaatattgaaggcaatattttactgctagcttgcaaagttatgagagtttaaaaaataaaatatatgaaaatatgtaaaaaaaaaaaaaaaaaaaaa

SEQ ID NO: 249 - DNA, 6633 bp, Homo sapiens
ttctccccgccccccagttgttgtcgaagtctgggggttgggactggaccccctgattgcgtaagagcaaaaagcgaaggcgcaatctggacactgggagattcggagcgcagggagtttgagagaaacttttattttgaagagaccaaggttgagggggggcttatttcctgacagctatttacttagagcaaatgattagttttagaaggatggactataacattgaatcaattacaaaacgcggtttttgagcccattactgttggagctacagggagagaaacaggaggagactgcaagagatcatttgggaaggccgtgggcacgctctttactccatgtgtgggacattcattgcggaataacatcggaggagaagtttcccagagctatggggacttcccatccggcgttcctggtcttaggctgtcttctcacagggctgagcctaatcctctgccagctttcattaccctctatccttccaaatgaaaatgaaaaggttgtgcagctgaattcatccttttctctgagatgctttggggagagtgaagtgagctggcagtaccccatgtctgaagaagagagctccgatgtggaaatcagaaatgaagaaaacaacagcggcctttttgtgacggtcttggaagtgagcagtgcctcggcggcccacacagggttgtacacttgctattacaaccacactcagacagaagagaatgagcttgaaggcaggcacatttacatctatgtgccagacccagatgtagcctttgtacctctaggaatgacggattatttagtcatcgtggaggatgatgattctgccattataccttgtcgcacaactgatcccgagactcctgtaaccttacacaacagtgagggggtggtacctgcctcctacgacagcagacagggctttaatgggaccttcactgtagggccctatatctgtgaggccaccgtcaaaggaaagaagttccagaccatcccatttaatgtttatgctttaaaagcaacatcagagctggatctagaaatggaagctcttaaaaccgtgtataagtcaggggaaacgattgtggtcacctgtgctgtttttaacaatgaggtggttgaccttcaatggacttaccctggagaagtgaaaggcaaaggcatcacaatgctggaagaaatcaaagtcccatccatcaaattggtgtacactttgacggtccccgaggccacggtgaaagacagtggagattacgaatgtgctgcccgccaggctaccagggaggtcaaagaaatgaagaaagtcactatttctgtccatgagaaaggtttcattgaaatcaaacccaccttcagccagttggaagctgtcaacctgcatgaagtcaaacattttgttgtagaggtgcgggcctacccacctcccaggatatcctggctgaaaaacaatctgactctgattgaaaatctcactgagatcaccactgatgtggaaaagattcaggaaataaggtatcgaagcaaattaaagctgatccgtgctaaggaagaagacagtggccattatactattgtagctcaaaatgaagatgctgtgaagagctatacttttgaactgttaactcaagttccttcatccattctggacttggtcgatgatcaccatggctcaactgggggacagacggtgaggtgcacagctgaaggcacgccgcttcctgatattgagtggatgatatgcaaagatattaagaaatgtaataatgaaacttcctggactattttggccaacaatgtctcaaacatcatcacggagatccactcccgagacaggagtaccgtggagggccgtgtgactttcgccaaagtggaggagaccatcgccgtgcgatgcctggctaagaatctccttggagctgagaaccgagagctgaagctggtggctcccaccctgcgttctgaactcacggtggctgctgcagtcctggtgctgttggtgattgtgatcatctcacttattgtcctggttgtcatttggaaacagaaaccgaggtatgaaattcgctggagggtcattgaatcaatcagcccggatggacatgaatatatttatgtggacccgatgcagctgccttatgactcaagatgggagtttccaagagatggactagtgcttggtcgggtcttggggtctggagcgtttgggaaggtggttgaaggaacagcctatggattaagccggtcccaacctgtcatgaaagttgcagtgaagatgctaaaacccacggccagatccagtgaaaaacaagctctcatgtctgaactgaagataatgactcacctggggccacatttgaacattgtaaacttgctgggagcctgcaccaagtcaggccccatttacatcatcacagagtattgcttctatggagatttggtcaactatttgcataagaatagggatagcttcctgagccaccacccagagaagccaaagaaagagctggatatctttggattgaaccctgctgatgaaagcacacggagctatgttattttatcttttgaaaacaatggtgactacatggacatgaagcaggctgatactacacagtatgtccccatgctagaaaggaaagaggtttctaaatattccgacatccagagatcactctatgatcgtccagcctcatataagaagaaatctatgttagactcagaagtcaaaaacctcctttcagatgataactcagaaggccttactttattggatttgttgagcttcacctatcaagttgcccgaggaatggagtttttggcttcaaaaaattgtgtccaccgtgatctggctgctcgcaacgtcctcctggcacaaggaaaaattgtgaagatctgtgactttggcctggccagagacatcatgcatgattcgaactatgtgtcgaaaggcagtacctttctgcccgtgaagtggatggctcctgagagcatctttgacaacctctacaccacactgagtgatgtctggtcttatggcattctgctctgggagatcttttcccttggtggcaccccttaccccggcatgatggtggattctactttctacaataagatcaagagtgggtaccggatggccaagcctgaccacgctaccagtgaagtctacgagatcatggtgaaatgctggaacagtgagccggagaagagaccctccttttaccacctgagtgagattgtggagaatctgctgcctggacaatataaaaagagttatgaaaaaattcacctggacttcctgaagagtgaccatcctgctgtggcacgcatgcgtgtggactcagacaatgcatacattggtgtcacctacaaaaacgaggaagacaagctgaaggactgggagggtggtctggatgagcagagactgagcgctgacagtggctacatcattcctctgcctgacattgaccctgtccctgaggaggaggacctgggcaagaggaacagacacagctcgcagacctctgaagagagtgccattgagacgggttccagcagttccaccttcatcaagagagaggacgagaccattgaagacatcgacatgatggacgacatcggcatagactcttcagacctggtggaagacagcttcctgtaactggcggattcgaggggttccttccacttctggggccacctctggatcccgttcagaaaaccactttattgcaatgcggaggttgagaggaggacttggttgatgtttaaagagaagttcccagccaagggcctcggggagcgttctaaatatgaatgaatgggatattttgaaatgaactttgtcagtgttgcctctcgcaatgcctcagtagcatctcagtggtgtgtgaagtttggagatagatggataagggaataataggccacagaaggtgaactttgtgcttcaaggacattggtgagagtccaacagacacaatttatactgcgacagaacttcagcattgtaattatgtaaataactctaaccaaggctgtgtttagattgtattaactatcttctttggacttctgaagagaccactcaatccatccatgtacttccctcttgaaacctgatgtcagctgctgttgaactttttaaagaagtgcatgaaaaaccatttttgaaccttaaaaggtactggtactatagcattttgctatcttttttagtgttaagagataaagaataataattaaccaaccttgtttaatagatttgggtcatttagaagcctgacaactcattttcatattgtaatctatgtttataatactactactgttatcagtaatgctaaatgtgtaataatgtaacatgatttccctccagagaaagcacaatttaaaacaatccttactaagtaggtgatgagtttgacagtttttgacatttatattaaataacatgtttctctataaagtatggtaatagctttagtgaattaaatttagttgagcatagagaacaaagtaaaagtagtgttgtccaggaagtcagaatttttaactgtactgaataggttccccaatccatcgtattaaaaaacaattaactgccctctgaaataatgggattagaaacaaacaaaactcttaagtcctaaaagttctcaatgtagaggcataaacctgtgctgaacataacttctcatgtatattacccaatggaaaatataatgatcagcaaaaagactggatttgcagaagttttttttttttttcttcatgcctgatgaaagctttggcaaccccaatatatgtattttttgaatctatgaacctgaaaagggtcagaaggatgcccagacatcagcctccttctttcaccccttaccccaaagagaaagagtttgaaactcgagaccataaagatattctttagtggaggctggatgtgcattagcctggatcctcagttctcaaatgtgtgtggcagccaggatgactagatcctgggtttccatccttgagattctgaagtatgaagtctgagggaaaccagagtctgtatttttctaaactccctggctgttctgatcggccagttttcggaaacactgacttaggtttcaggaagttgccatgggaaacaaataatttgaactttggaacagggttggaattcaaccacgcaggaagcctactatttaaatccttggcttcaggttagtgacatttaatgccatctagctagcaattgcgaccttaatttaactttccagtcttagctgaggctgagaaagctaaagtttggttttgacaggttttccaaaagtaaagatgctacttcccactgtatgggggagattgaactttccccgtctcccgtcttctgcctcccactccataccccgccaaggaaaggcatgtacaaaaattatgcaattcagtgttccaagtctctgtgtaaccagctcagtgttttggtggaaaaaacattttaagttttactgataatttgaggttagatgggaggatgaattgtcacatctatccacactgtcaaacaggttggtgtgggttcattggcattctttgcaatactgcttaattgctgataccatatgaatgaaacatgggctgtgattactgcaatcactgtgctatcggcagatgatgctttggaagatgcagaagcaataataaagtacttgactacctactggtgtaatctcaatgcaagccccaactttcttatccaactttttcatagtaagtgcgaagactgagccagattggccaattaaaaacgaaaacctgactaggttctgtagagccaattagacttgaaatacgtttgtgtttctagaatcacagctcaagcattctgtttatcgctcactctcccttgtacagccttattttgttggtgctttgcattttgatattgctgtgagccttgcatgacatcatgaggccggatgaaacttctcagtccagcagtttccagtcctaacaaatgctcccacctgaatttgtatatgactgcatttgtgggtgtgtgtgtgttttcagcaaattccagatttgtttccttttggcctcctgcaaagtctccagaagaaaatttgccaatctttcctactttctatttttatgatgacaatcaaagccggcctgagaaacactatttgtgactttttaaacgattagtgatgtccttaaaatgtggtctgccaatctgtacaaaatggtcctatttttgtgaagagggacataagataaaatgatgttatacatcaatatgtatatatgtatttctatatagacttggagaatactgccaaaacatttatgacaagctgtatcactgccttcgtttatatttttttaactgtgataatccccacaggcacattaactgttgcacttttgaatgtccaaaatttatattttagaaataataaaaagaaagatacttacatgttcccaaaacaatggtgtggtgaatgtgtgagaaaaactaacttgatagggtctaccaatacaaaatgtattacgaatgcccctgttcatgtttttgttttaaaacgtgtaaatgaagatctttatatttcaataaatgatatataatttaaagtt

SEQ ID NO: 250 - DNA, 4125 bp, Homo sapiens
cgactgggccaggcgccggggcaggaagggaggcggccgccgtgccattcttaaaggcgcccgagtgtaggcgacaggccgctgacggccggaaggaaaatgagtgagtctttggttgtttgtgatgttgccgaagatttagtggaaaagctgagaaagtttcgttttcgcaaagaaacgaacaacgctgctattataatgaagattgacaaggataaacgcctggtggtactggatgaggagcttgagggcatttcaccagatgaacttaaagatgaactacctgaacgacaacctcgcttcattgtgtatagttataaatatcaacatgatgatggaagagtttcatatcctctgtgctttattttctccagtcctgttggatgtaagcctgaacaacagatgatgtatgctggaagtaagaataagctagtccagacagctgaactaaccaaggtatttgaaataagaaataccgaagacctaactgaagaatggttacgtgagaaacttggattttttcactaatgtgaacttctgtgtttctaaagtatttatgtattaacctgaccatactggaatcagacataaatacttatttatgcctaaaaatgcactgttacttacagtttgtttcctgcagtaaagaaaaattcttcatttgtgcaaaatttgaacaaagaggaaatcatcttcatagtaatgaaactttgtaaagtgtttccttatattggtaattgttaggtggactacttttctccagggactttttgcactcttgtgactaatttctataacttatggttcggaatttgttactatttacagacaccattggaaagtggatatattagattgtgagagacaacagttgcctccttttgacaaatactggatattagcagtttatttatgaaaatagcgtattatcacttgtcaaatcattgaaattcatttggggtcaaagacttgagtgacccagtattgagccatgaataatttagtgtaacctgtattacaagtacattgatgaattctgtatcttctttggtttcctgtatctttttaatcaagtctagaaactatgttcatcagtcactcatttttaaggtcgggagttagattttatgatagaattatgactgttagcttttctccttatagcatcttagtcttagaaattggtgggttgtaataatcaagggcttcattccttttatgtcatttctagacagttttgaatctaggttaataacactttatttataaagcacctcaatgtcctgtgaacactaattattttaaatgtgttaatactgtgcctttgatttgttagctttaaagttagtttaagacttttacactgccagtattccacatttggtgaaattaatacttttttaaagggtccaaataaaataattttctaatgtgtatatctgaaatttgtaataaaatcaacttcatattttaaaaattccaactatctgcttgcattggtgaatatatggcagtcgagagttataattttgggtatacttgtggttagttttgtgccataggaaaaaattatcttaaaactttggccatagttaataacattaacacttcaatagcaatcacatcttatatcctaaatgtcagaagatattctgaactggatgcctgaatagttaactaaaccagtcttgttagatgatggtactcttggcataaagcgaggattctgatatttggcatacttgtaaaaacaaatacataagtaaccattgaacattaatttgataataggtctagagactctaaaaactaaccaaacttggtgagtgtattcttatattaagaatatcttagtcatctcaaaactagcaaaatttaaattttggcatgttttccattcatatgttctttgcattttatttttgaggtttctgtgagaagtaaagatagttggaatttttgcgatattgaatagaacatcttctgttcccaacactgtttggcttcactaatttagaagtcaggaagcaatagaaagttggagatgaggaagtgctagagtaggtgtttgttttggttcttggagggaaaagattctttattccaatttccagagagaagagaaaactcacccaggaagtttaaaaattctttaaacaggtattttgatattggagaataacatgcatataattctgtaggaatgcacatgtaatccaagtgagtggagagtgtttttaatgtttttgaatgaaggaaatgaggttttgtttcacctgttttgcagcagtaagagaaactagtgctgcaagaatgtattttttaatgaagttccttattttgtcttgcatgttttagttttgcttatttttaaatttggaggtcctccataatgtcagataatattgacctgccatacgttagcactcttagttccgctactgtctttaacaggagcaaagagctgtgataaaccatgcttttttgagcttgtctgactcctaattaataacatgtttttggcaagacaacagattgaggttagaggatcagtaggacatttttattccatctgtcctatggggaaatttacaaatcccgtgctctaaaatgttctcaaacatttatatagatttccctttcatcttactaaattttgcattgttcttttcaagtatgtttcgtatttactgtctttttttctgccatttcccaaataataactccagatttcataattccagtttttacattccgttatctttctggtacaaccattcccattcagccttaaatctgagtcctttttagcagcaacttttttcctgggatcctccttcgtggtcttctaagtcagtgttagttttgaaatttttggccctgcataagttctgcatagcatctaatgtcaaaatagaaccaactggtaatcacagtattatttagtgtggtttccatgacaacaaaaatacatacgaagaaaacttctcaggttactatgctgaaattccaaaatgtctgagttttgaatagtgatcactttgttctggtattgacgcaattatattaggaaaaaagttggttgactgtttttgtttaattgacttctaaaatgttcaaattgtctagttctaaaagtttactaaatgcctagtgcagttaaacatactcttgtttaagtgtgtgttgctaaattttttactgtcattactaaataatctgtgtggcaaaatgtgtgtcagcacttttccctccttttttatctcctattttcaggagtcaaatgtagccataaactgtatccttgtctgacactttagctaaaaatttccagttaggggagtttattgccaaattaaatttggctgttccccccaacccatatagatattaaggaaggtgtacttaaaaaatgtttggactgcttttaaaacctgagcaatgtcattaatccatatgtggactagtgatgaatagatattttcataagagtttaaatgctgatatttggtggaagtagagagtaactcatattctatcaattcaagtattcttactatggttgctttccctatttgttcaatagactgataatactggaatttatagagtttgagccattacaacttttgtgaggatgtgtttcaaacatttctggacaaatcttattttgtatttctggaagaatgtagtaatcttctagaccgcttaaaaccaatgctcccaagctgaatattcttgagaaatttgtttttattatgccatttgacatttcaaatcagtgctcatatacagtaaacttgtgatagaaattgtattttattgctttttggattataattcatataaatataattacttgaatattgtttgagatcattaacatgccagggcagttcccactgatttagatggtccaagataatctcattcaggaggcttgaaacattaatggtttagtcttgtgaattttaacagttctctgtcatcgtttaacaaaaccaacaactgacacaactccttaagctgtggtttcagtctctgctagttcatattgcatgtttattttggacagtcttttgttaagcatggtgcttgtactggtttaaataaaatgttaacatt

SEQ ID NO: 251 - DNA, 31 bp, Artificial Sequence
  Description of Artificial Sequence primer
gggatccaccggataatctagagcggccgct

SEQ ID NO: 252 - DNA, 35 bp, Artificial Sequence
  Description of Artificial Sequence primer
gatcagcggccgctctagattatccggtggatccc

SEQ ID NO: 253 - DNA, 21 bp, Artificial Sequence
  Description of Artificial Sequence primer
actacctgagcacccagtccg

SEQ ID NO: 254 - DNA, 22 bp, Artificial Sequence
  Description of Artificial Sequence primer
cggacttctacagctcgtccat

SEQ ID NO: 255 - DNA, 23 bp, Artificial Sequence
  Description of Artificial Sequence primer
gaccgctatcaggacatagcgtt

SEQ ID NO: 256 - DNA, 24 bp, Artificial Sequence
  Description of Artificial Sequence primer
aagaactcgcaagaaggcgataga

SEQ ID NO: 257 - DNA, 24 bp, Artificial Sequence
  Description of Artificial Sequence primer
gctcaccatggatgatgatatcgc

SEQ ID NO: 258 - DNA, 22 bp, Artificial Sequence
  Description of Artificial Sequence primer
gacctggccgtcaggcagctcg